(19) United States
(12) Patent Application Publication
(10) Pub. No.: US 2019/0015352 A1
(43) Pub. Date: Jan. 17, 2019

(54) Applicators & Patches for Dermal & Transdermal Treatment Material & Drug Delivery, Methods of Making Them, & Methods of Use

(71) Applicant: Pendleton Brewster Wickersham, San Antonio, TX (US)

(72) Inventor: Pendleton Brewster Wickersham, San Antonio, TX (US)

(21) Appl. No.: 16/027,701

(22) Filed: Jul. 5, 2018

Related U.S. Application Data
(60) Provisional application No. 62/604,652, filed on Jul. 14, 2017.

(51) Int. Cl. A61K 9/70

(52) U.S. Cl. CPC A61K 9/7084; A61F 2013/0296

(57) ABSTRACT

Dermal and transdermal patches (also called “applicators”) for delivering treatment material. In certain aspects, a treatment patch for applying treatment material to the skin of living beings, for example, a mammal or animal, including, but not limited to human beings. In at least some embodiments, such a patch includes a main body made up of a plurality of interconnected subunit patches with open space between patches. Adjacent patches are connected with one, two, three or more connectors connected to each adjacent patch and extending between them. The connectors can be of the same size and thickness as the interconnected patches (often herein “subunit patches”). In size, the connectors can be of the same length, width, height and area as the patches. The patches can all be the same, or different patches can be used as parts of the same main body.

### CROSS REFERENCES TO RELATED APPLICATIONS

This application claims the benefit under Title 35 United States Code § 119(e) of U.S. Provisional Patent Application Ser. No. 62/604,652; Filed: Jul. 14, 2017; the full disclosure of Which is incorporated herein by reference.

### BACKGROUND OF THE INVENTION

### 1. Field of the Invention

The present invention is directed to applicators and patches for application to skin for dermal and transdermal treatment material delivery and drug delivery. In certain aspects, such patches are within a network, web, net, lattice-like stricture, or reticulated multi-component structure with open areas. In certain aspects, such patches include multiple material delivery portions interconnected by multiple connection parts. Such patches in which drug delivery portions and/or connection portions are flexible, separable, tearable, frangible, stretchable, and/or elastic, and in certain aspects, such patches which facilitate effective adherence to skin. In certain aspects, methods for making transdermal drug delivery patches and methods for using such patches.

### 2. Description of the Related Art

There are a wide variety of known patches used for transdermal drug delivery. There are a variety of problems associated with known dermal and transdermal patches. One problem is getting a patch to effectively adhere to skin. Such a problem is often made worse by the movement of the person or animal on which a patch is being used. Another problem is how to efficiently transmit treatment material from a patch to that area, and to only that area, that is to be treated.

A further problem is the application of a proper dose of treatment material without applying an insufficient amount and without applying too much. Problems are also encountered with a patch body of a particular unchangeable set shape and configuration which does not conform to a skin area or a part of a body, particularly with bending and movement of the skin or body.

Skin requires exposure to the air for health and to allow for evaporation of sweat. The stratum corneum layer of the skin can be damaged when sweat accumulates under a barrier, for example, an impermeable barrier which is a layer of a patch. Excess sweating under a patch (especially in warmer climates) can also lead to loss of adherence. This can lead to irritation of the skin and loss of its normal protective function. In some cases, in which a dermal or transdermal patch is used, this also can cause increased absorption of treatment material or medication beyond an intended dose as well as increased risk of infection.

Accordingly, there exists need for effective and efficient patches for dermal and transdermal material and drug delivery. There is a need for such patches that effectively and continuously adhere to skin while delivering material and drugs and to such patches that effectively accommodate movement of the body and of the skin to Which a patch is adhered.

### SUMMARY OF THE INVENTION

The present invention, in certain aspects and embodiments, discloses dermal and transdermal patches (also called “applicators”) for delivering treatment material. In certain aspects, the present invention discloses a treatment patch for applying treatment material to the skin of living beings, for example, a mammal or animal, including, but not limited to human beings. In at least some embodiments, such a patch includes a main body made up of a plurality of interconnected subunit patches with open space between patches. Adjacent patches are connected with one, two, three, four, five, six, or more connectors connected to each adjacent patch and extending between them. The connectors can be of the same size and thickness as the interconnected patches (often herein “subunit patches”), or not. In size, the connectors can be of the same length, width, height and area as the patches, or not. The patches can all be the same, or different patches can be used as parts of the same main body.

Both patches and connectors can be of any desired shape, size and configuration. This makes possible the provision of open spaces between patches of any desired shape, area, and volume. Connectors between patches may be made of any desired material. In some aspects, the connectors are made of flexible material. In some aspects, the connectors are made of elastic or stretchable material. Using such material or materials, makes it possible for a patch according to the present invention to stay in place when a person or animal to whom the patch has been applied, moves, bends, or is moved, and in some circumstances, allows the patch to conform more closely to an area of skin or to a body part. A patch with such connectors can accommodate such movement or bending and still remain adhered to skin. Using such connectors also makes it possible for a patch to remain in place if the person or animal is subjected to touch, stress, or impact by something else, or if the patch itself is touched, subjected to stress, moved, impacted, pushed, or pulled. Connectors may be in touch with and/or adhered to skin, or not. Connectors may be formed integrally of or adhered to subunit patches. For such adherence, any suitable adhesive material may be used,

This invention also discloses the use of string-like connectors, thin or thick, between patches, for example, but not limited to, string, twine, fibers, filaments, wires, and the like, as well as very thin, small cross-sectional area connectors, made of any material disclosed herein for a connector. Such string-like connectors make possible the orientation and placement of adjacent subunit patches in contact with each other or at any desired distance from each other (depending on the length of the string-like connectors) and at any angle with respect to adjacent patches. Such connectors can also facilitate patch conformation to varying or non-flat shapes of skin or of body parts. Also, by using multiple, relatively smaller subunit patches, it is possible to reduce the effects of differences in shape between the lower surface of a patch and the area of skin over which the patch is placed, thus reducing strain on the overall main patch. In one aspect, for example, a plurality of spaced-apart subunit patches has more patch surface adhering to a projecting skin area than a single relatively larger patch would.

It is within the scope of this invention to make a main patch with a body with multiple subunit patches in which the adjacent subunit patches of the body are separable from each other so that any desired subunit patch or number of patches may be separated from a body of a plurality of subunit patches. Connectors in or on the body used between patches may be made of easily separated, easily torn, or easily cut material. Separation can be facilitated by making the connectors of a desired thickness, using connectors with perforations, or using connectors with scoring that renders the material more easily separated.

In certain aspects, the present invention provides a sheet or a layer (sometimes called a “release layer” or “release liner”) with multiple subunit patches thereon which are removable from the sheet or layer so that, as removed, there are openings between the removed patches and connectors. Therefore, a removed portion of the sheet or layer provides a body with multiple subunit patches thereon of any desired number. The removed portion or portions may then be applied to skin with any desired spacing between portions.

The present invention provides, in certain aspects, a patch with a top, backing layer, support, or cover with one or more holes or openings through which treatment material may be applied to drug-containing material of the patch (or to material within the patch that has therein or thereon, treatment material. This can be an impermeable layer. In one aspect, at some time after a patch is applied to skin, treatment material is introduced through the hole or holes tor treatment of a person or animal, or for cosmetic purposes. In one particular aspect, as is true for any embodiment of the present invention, the treatment material is: a drug, drugs, or medicine; an essential oil; vinegar; water; nicotine; caffeine; nitroglycerine; anti-fungal material; antiviral material; anti-bacterial material; antibiotic material; cosmetic material; and/or odor masking material; or some combination of any two or more of these. One, some, or all subunit patches of a main patch may have such a hole or holes. In certain aspects, the present invention provides a patch as any described herein to which is applied cosmetic material such as perfume or cologne. The cosmetic material can be applied to the patch before application to skin, after application to skin, or both. In other aspects, repellent material or de-scenting material is applied using the patch.

For an adhesive for adhesion of a patch according to the present invention to skin, any suitable known adhesive may be used. Some or all of a surface of a patch for adhesion to skin may have adhesive material; some or all of a surface of a connector for adhesion to skin may have adhesive material. Adhesive may be only on patch portions, only on connector portions, or on both. In certain aspects, the connectors do not touch skin when the patch is applied and the connectors have no adhesive thereon.

With appropriate sizing and shaping according to the present invention, the present invention provides new and nonobvious improvements to known patch systems by providing for the connection of multiple known patches by connectors, according to the present invention, to provide a main patch (with multiple subunit patches) with open area between patches and/or with at least one or a plurality of connectors between the multiple subunit patches.

Accordingly, the present invention includes features and advantages which are believed to advance transdermal drug delivery technology and the technology of patches used for this. Characteristics and advantages of the present invention described herein, and additional features and benefits will become known to and appreciated by those skilled in the art who have the benefit of the new and nonobvious teachings of the present invention, upon consideration of the following detailed description of certain embodiments and referring to the accompanying drawings.

Certain embodiments of this invention are not limited to any particular individual feature or aspects disclosed here hut include combinations of them distinguished from the prior art in their structures, functions, designs, configurations, methods of use, and/or results achieved. Features and aspects of the invention have been broadly described so that the detailed descriptions that follow may be better understood, and in order that the contributions of this invention to the arts and to technology may be better appreciated. There are, of course, additional aspects of the invention described below, and which may be included in the subject matter of the claims to this invention

Those skilled in the art who have the benefit of this invention, its teachings, and suggestions will come to know that the conceptions of this disclosure may be used as a creative basis for designing other structures, methods and systems for carrying out and practicing the present invention. The claims of this invention are to be read to include any legally equivalent structures, systems, devices or methods which do not depart from the spirit and scope of the present invention.

The present invention and its diverse embodiments recognize and address certain needs and provide solutions to problems and a satisfactory meeting of those needs in its various possible embodiments and equivalents thereof. To one of skill in this art who has the benefits of this invention's realizations, teachings, disclosures, and suggestions, other purposes and advantages will become known from the following description of certain embodiments, given for the purpose of disclosure, when taken in conjunction with the accompanying drawings. The detail in these descriptions is not intended to thwart this patent's object to claim this invention no matter how others may later disguise it by variations in form, changes, or additions of further improvements. It will be understood that the various embodiments of the present invention may include one, some, or any possible combination of the disclosed, described, and/or enumerated features, aspects, elements, and/or improvements and/or technical advantages in claims to this invention.

As used herein, the term “and/or” includes any and all combinations of one or more of the associated listed items. Spatially relative terms, such as “below”, “tower”, “upper” and the like, can be used herein for ease of description to describe one element or feature's relationship to another element or feature as illustrated in the figures. it will be understood that the spatially relative terms are intended to encompass different orientations of the device in use or operation in addition to the orientation depicted in the figures. For example, if a structure in the figures is turned over, elements described as “below” or “lower” relative to other elements or features would then be oriented “above” relative to the other elements or features. Thus, the exemplary term “below” can encompass both an orientation of above and below. The structure can be otherwise oriented (rotated 90 degrees or at other orientations) and the spatially relative descriptors used herein interpreted accordingly.

### BRIEF DESCRIPTION OF THE DRAWINGS

The invention may be understood by reference to the following descriptions taken in conjunction with the accompanying drawings, in which, like reference numerals identify like elements. These drawings illustrate certain embodiments of the present invention and are not to be used to improperly limit the scope of the invention. which may have other equally effective or legally equivalent embodiments. In the appended figures, similar components and/or features may have the same numerical reference label. Various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix,

FIG. 1A is a top view of a patch according to the present invention

FIG. 1B is a cross section view along line A-A of FIG. 1A.

FIG. 1C is a cross section view of a patch according to the present invention.

FIG. 1D is a cross section view of a patch according to the present invention,

FIG. 1E is a cross section view of a patch according to the present invention.

FIG. 1F is a cross section view of a patch according to the present invention.

FIG. 1G is a cross section view of a patch according to the present invention.

FIG. 1H is a cross section view of a patch according to the present invention

FIG. 1J shows the patch of FIG. 1H in place on skin.

FIG. 1K is a cross section view of a patch according to the present invention.

FIG. 1L shows the patch of FIG. 1K in place on skin.

FIG. 1M is a cross section view of a patch according to the present invention.

FIG. 1N shows the patch of FIG. 1M in place on skin.

FIG. 1P is a cross section view of a patch according to the present invention.

FIG. 1Q shows the patch of FIG. 1P in place on skin.

FIG. 1R is a cross section view of a patch according to the present invention on skin.

FIG. 1S shows a patch according to the present invention on skin.

FIG. 2A is a cross section view of a patch according to the present invention.

FIG. 2B is a cross section view of a patch according to the present invention.

FIG. 2C is a cross section view of a patch according to the present invention.

FIG. 2D is a cross section view of a patch according to the present invention.

FIG. 3 is a top view of various possible shapes, as viewed from above, for patches according to the present invention.

FIG. 4 is a top view of various possible shapes, as viewed from above, for connectors according to the present invention.

FIG. 5A is a top view of a patch according to the present invention.

FIG. 5B is a top view of patches from the patch of FIG. 5A as applied to skin.

FIG. 5C is a top view of patches from the patch of FIG. 5A as applied to skin.

FIG. 6 is a top view of a patch according to the present invention.

FIG. 7 is a top view of a patch according to the present invention.

FIG. 8 is a top view of a patch according to the present invention.

FIG. 9 is a top view of a patch according to the present invention.

FIG. 10 is a top view of a patch according to the present invention.

FIG. 11 is a top view of a patch according to the present invention.

FIG. 12A is a top view of a patch according to the present invention.

FIG. 12B is a cross section view of an embodiment of subunit patches of the patch of FIG. 12A.

FIG. 13A is a top view of a patch according to the present invention.

FIG. 13B is a cross section view of an embodiment of subunit patches of the patch of FIG. 13A.

FIG. 14A is a top view of a patch according to the present invention.

FIG. 14B is a cross section view of an embodiment of subunit patches of the patch of FIG. 14A.

FIG. 14C is a cross section view of an embodiment of subunit patches of the patch of FIG. 14A.

FIG. 15A is a top view of a patch according to the present invention.

FIG. 15B is a cross section view of an embodiment of subunit patches of the patch of FIG. 15A.

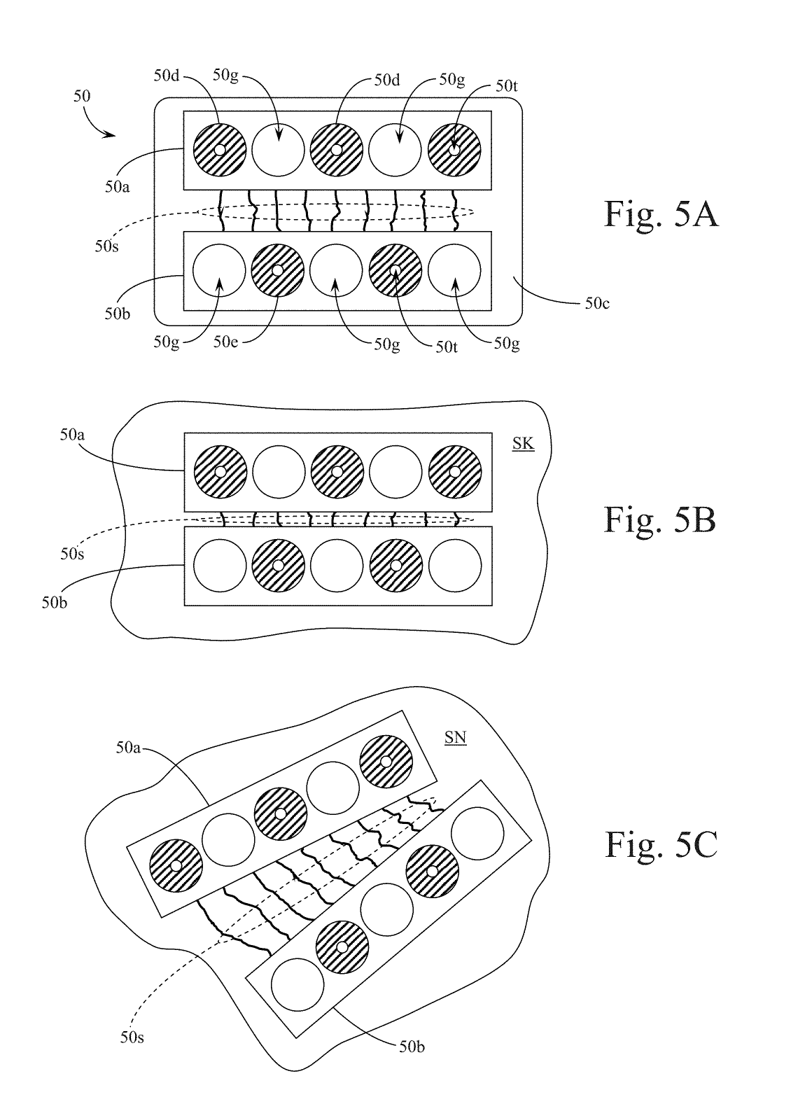

FIG. 5C is a cross section view of an embodiment of subunit patches of the patch of FIG. 15A.

### DETAILED DESCRIPTION OF THE PREFERRED EMBODIMENTS

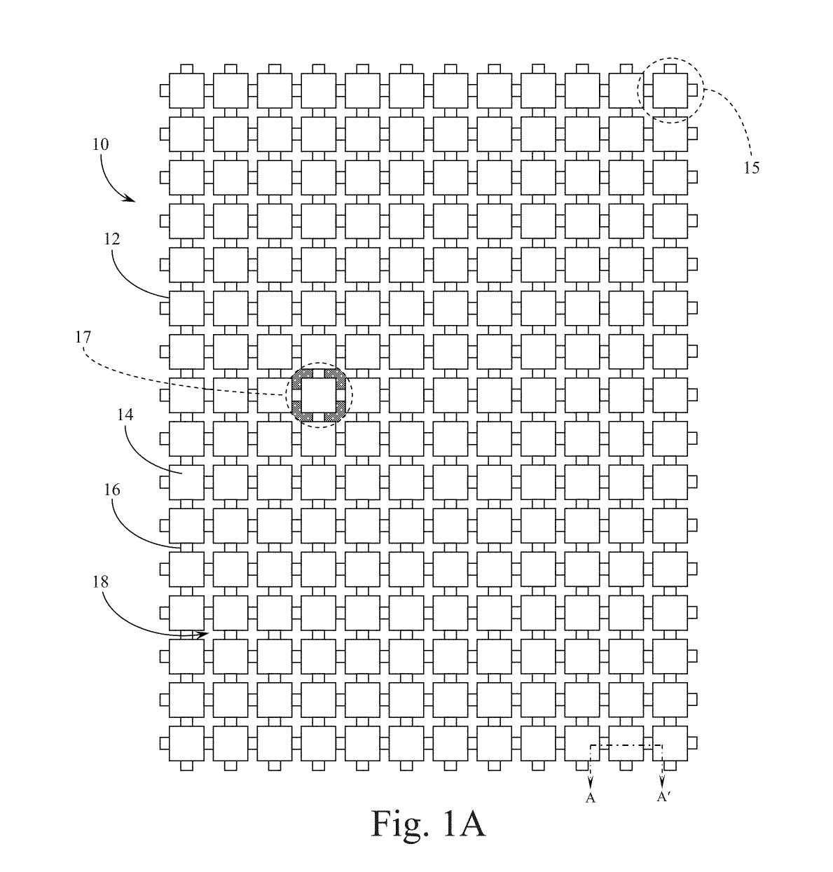

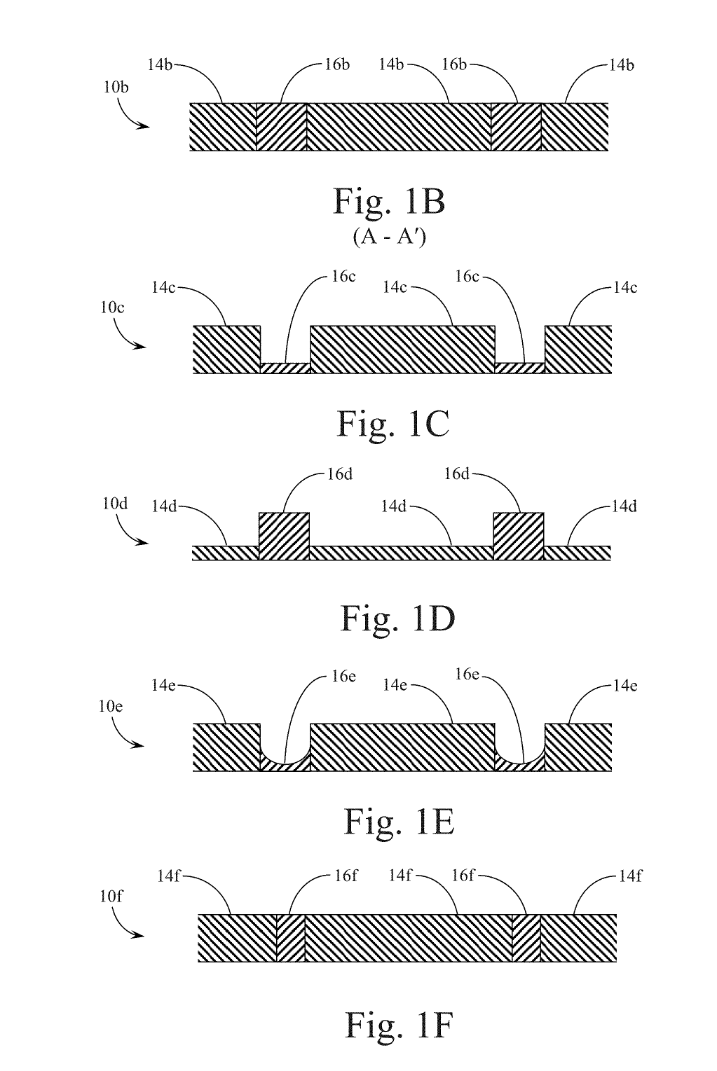

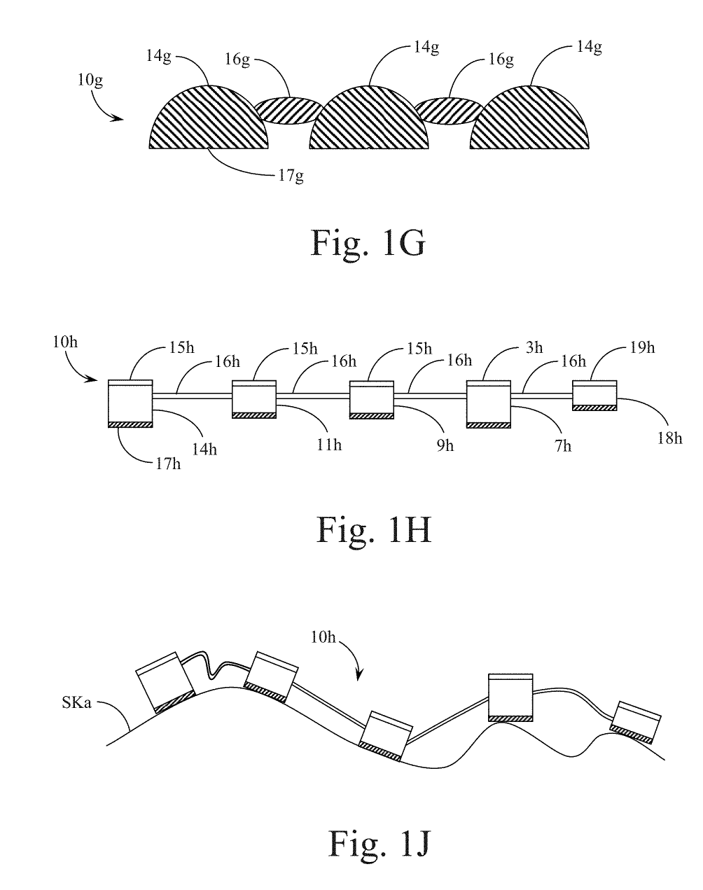

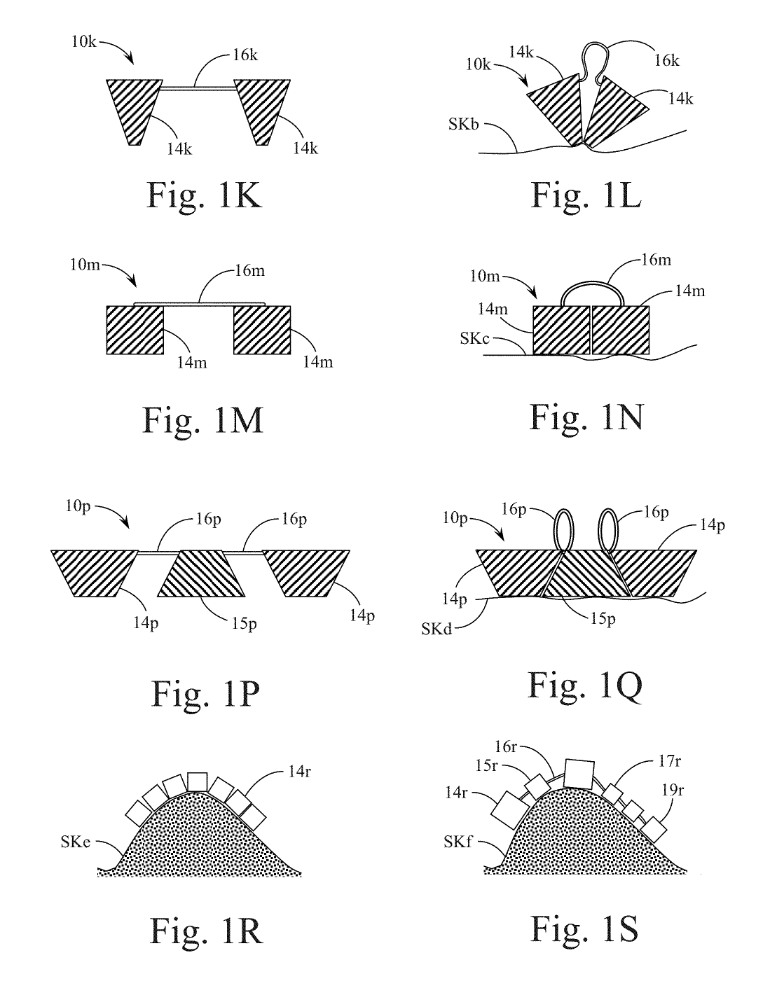

FIGS. 1A and 1B show a main patch 10 according to the present invention which has a body 12 that includes multiple subunits, each of which is a subunit patch 14 interconnected by multiple connectors 16, with open areas 18 around each subunit patch 14 defined by surfaces of the subunit patches and connectors. Within broken line circle 15 the crosshatched area indicates a subunit patch 14 and its connectors 16. Within broken line circle 17 the crosshatched area indicates an open area 18 around a patch. Each subunit patch may be used for application to the skin of a person or of an animal for dermal or transdermal treatment. The connectors and the subunit patches may be of any desired size, shape, and thickness; and, as shown, are of the same thickness. It is within the scope of this invention for all subunit patches 14 to be of different size and/or shape; or, as shown, for them all to be substantially the same. It is within the scope of this invention for all open areas 18 to be of different size and/or shape; or, as shown, for them all to be substantially the same.

FIG. 1C shows (partially) a main patch 10c according to the present invention which is like the main patch 10 of FIG. 1A and looks similar to the main patch 10 as viewed from above, but which has connectors 16c between subunit patches 14c. It is within the scope of the present invention for connectors to be separate parts which are connected to subunit patches or, as shown in FIG. 1C, material of the connectors and the subunit patches are integral with each other. It is within the scope of the present invention for the connectors, for example as shown in FIG. 1B, to be of the same thickness as the subunit patches or, as shown in FIG. 1C, the connectors can be of a thickness less than that of the subunit patches. For any of the connectors 16c and/or the subunit patches 14c (as is true for any connector and any subunit patch of any of the embodiments of FIGS. 1B-1H), there may be adhesive on the bottom surface of the connector and/or subunit patch, or not. The bottom of the patch 10c is the bottom (the lowermost surface) as shown in FIG. 1C (as is true for the bottom of the patches of FIGS. 1B and 1D-1H).

FIG. 1D shows a main patch 10d according to the present invention which is like the main patch 10 of FIG. 1A, but which has connectors 14d between subunit patches 14d. The connectors 16d are thicker than the subunit patches 14d. FIG. 1E shows a main patch 10e according to the present invention which is like the main patch 10 of FIG. 1A, but which has crescent shaped connectors 16e (crescent shaped when viewed as in FIG. 1E) between subunit patches 14e. Such a crescent shape provides adequate connection of connectors to sides of a subunit patch, and a lowermost part that is relatively thin to facilitate separation of the subunit patches, if desired, and/or cutting, severing, or tearing of the connectors. FIG. 1F shows a main patch 10f according to the present invention which is like the main patch 10 of FIG. 1A, but which has connectors 16f between patches 14f. The cross section of the connectors 16f, as shown in FIG. 1F, is rectangular.

It is within the scope of the present invention to use subunit patches of any desired dimensions. FIG. 1G shows a main patch 10g according to the present invention which is like the main patch 10 of FIG. 1A, but which has subunit patches 14g with a dimension that is more than six times greater than a dimension of connectors 16g. FIG. 1H shows a main patch 10h with subunit patches 14h, 11h, 9h, 7h, and 18h connected by connectors 16h. The subunit patches have a top layer 15h, 3h, or 19h. As shown in FIG. 1J, as applied to an area of skin SKa, the connectors 16h can flex, contract, stretch, bend, move, and/or expand so that the subunit patches can be adhered to a desired skin part.

FIG. 1K shows a main patch 10k with subunit patches 14k. It is to be understood that it is within the scope of the present invention for a main patch according to the present invention to have any desired number of subunit patches 14k, including any number shown in any drawing figure herein or any number mentioned in the text herein. As shown in FIG. IL, the connectors 16k can flex, contract, bend, move, and/or stretch so that the subunit patches 14k can be adhered to a desired skin part, can be finally positioned in contact with each other as shown (or very near each other), and all or part of the connector can move to a location not between two subunit patches which the connector connects. In certain aspects, the pointed or cone shape of the lower part of the subunit patches 14k facilitates emplacement of the patches at or above a non-flat skin part of the area of skin SKb.

FIG. 1M shows a main patch 10m with subunit patches 14m. It is to be understood that it is within the scope of the present invention for a main patch according to the present invention to have any desired number of subunit patches 14m, including any number shown in any drawing figure herein or any number mentioned in the text herein. As shown in FIG. 1N, the connectors 16m can flex, move, and/or bend so that the subunit patches 14m can be adhered in contact with each other at a desired skin part of a skin area SKe, can be finally positioned with one part or one side of one subunit patch in contact with a side of an adjacent subunit patch, and the connector can move to a location not between the two subunit patches, so it does not prevent or restrict their contact.

It is within the scope of the present invention to provide a patch with adjacent subunit patches whose sides correspond to each other and whose sides are inclined, slanted, canted, or curved to provide contact surfaces for adjacent subunit patches when they are applied to an area of skin. Connectors for such subunit patches can end up above the subunit patches, between them, or both. FIG. 1P shows a main patch 10p with subunit patches 14p, 15p, and 14p connected by connectors 16p. In position on a skin area SKd, as shown in FIG. 1Q, sides of the subunit patches 14p and 15p are in contact (the left pair as shown in FIG. 1Q) and sides of the subunit patches 15p and 14p are in contact (the right pair in FIG. 1Q). Among other things, using such sides as those of the subunit patches 14p and 15p stabilizes the subunit patches in place and inhibits or prevents their unwanted movement on the skin.

FIG. 1R illustrates that it is within the scope of the present invention to provide a main patch with subunit patches 14 (seven shown but any number possible) which can be located above/on a non-flat skin area of skin SKe. The subunit patches, as shown, can be finally positioned on the skin so that they are in contact with each other and with the skin.

FIG. 1S illustrates that it is within the scope of the present invention to provide a main patch with subunit patches 14s, 15s, 17s, and 19s (any number of any type possible) which can be located above/on a non-flat skin area of skin SKf. The subunit patches, as shown, can be finally positioned on the skin so that they are spaced-apart from each other with connectors 16s between them, and in contact with the skin.

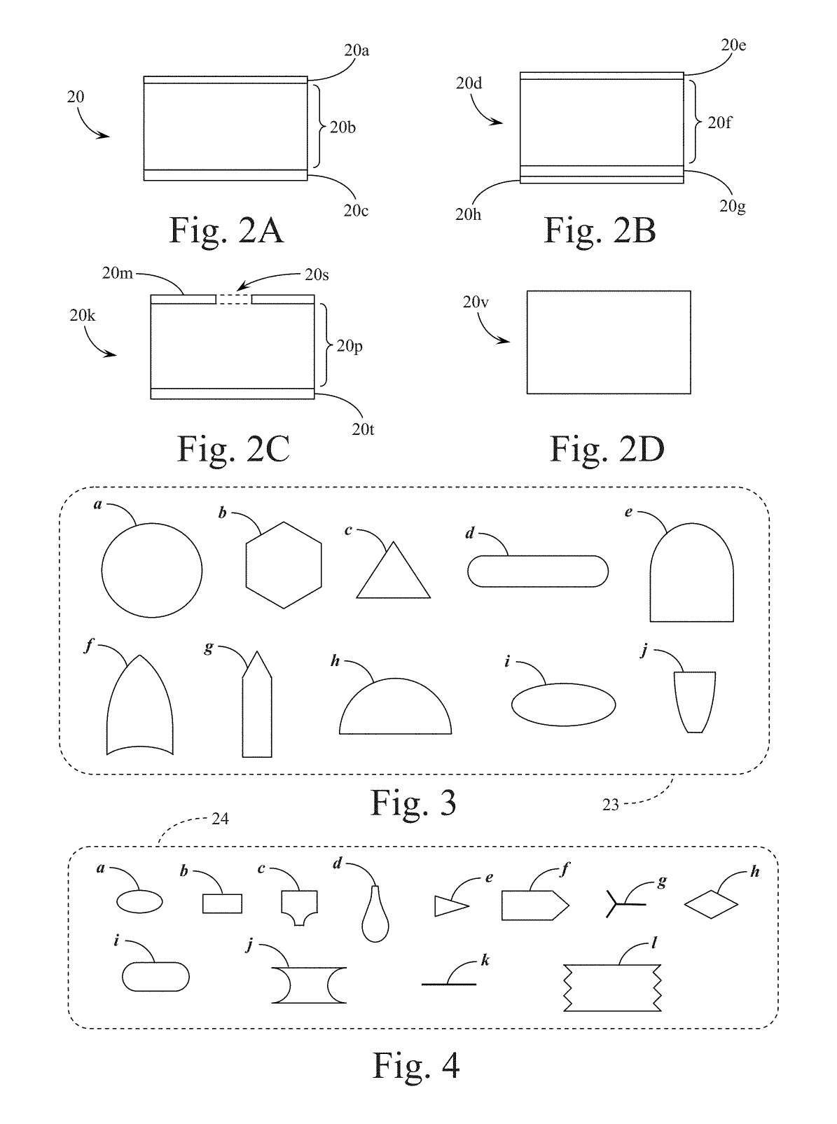

FIG. 2A shows in cross section a structure 20 for a patch according to the present invention (which may be the structure for any patch or subunit patch disclosed herein). A top, cover, support, or backing layer 20a overlies an adhesive layer 20b. The adhesive layer 20b, prior to application of the patch to skin, is releasably disposed on a release layer or release liner 20c, from which the patch can be easily removed or peeled away. As desired, the backing layer may be impermeable. The adhesive of the adhesive layer adheres to the lower surface of the backing layer 20a. Treatment material, for example, but not limited to a drug, may be in or on the adhesive layer, or both.

FIG. 2B shows in cross section a structure 20d for a patch according to the present invention (which may be the structure for any patch or subunit patch disclosed herein). A top, cover, support, or backing layer 20e overlies a treatment material layer 20f which is on an adhesive layer 20g. The adhesive layer 20g, prior to application of the patch to skin, is releasably disposed on a release layer or release liner 20h, from which the patch can be easily removed or peeled away. As desired, the backing layer may be impermeable. FIG. 2C shows in cross section a structure 20k for a patch according to the present invention (which may be the structure for any patch or subunit patch disclosed herein). The patch 20k has a top layer 20m over an adhesive layer 20p which is on a release layer 20t. The top layer 20m has hole 20s therethrough which provides a location for the introduction of treatment material into the adhesive layer 20p and/or for ventilation of the adhesive layer. Optionally, a layer of treatment material, for example like the layer 20g of FIG. 2B, may be used (above the adhesive layer) with treatment material therein and/or thereon.

FIG. 3 shows as top views a variety of shapes for subunit patches according to the present invention. It is to be understood that in a network, grid, layer, sheet, or reticulated member that has subunit patches with any shape shown, the subunit patches may be oriented in any direction and/or at any angle, including, but not limited to, as shown in FIG. 3. In a single main patch, it is within the scope of the present invention to use subunit patches of different shapes. 4 shows as top views a variety of shapes for connectors according to the present invention. It is to be understood that in a network, grid, layer, sheet, or reticulated member that has subunit patches and a connector or connectors with any shape shown, the connector or connectors may be oriented in any direction and/or at any angle, including, but not limited to, as shown in FIG. 4. In certain aspects, the connectors labeled “g” and “k” are string-like connectors. In a single main patch, it is within the scope of the present invention to use connectors of different shapes and/or to use a combination of string-like connectors and connectors that are not string-like.

FIG. 5A shows a main patch 50 according to the present invention with subsidiary patches 50a and 50b on a release liner 50c. String-like connectors 50s connect the subunit patches 50a, 50b together. Subsidiary patch 50a has subunit patches 50d. and subsidiary patch 50b has subunit patches 50k. The subsidiary patches have openings 50g. The subunit patches may be as any subunit patch disclosed herein. FIG. SB shows the subsidiary patches 50a and 50b removed from the release liner 50c and applied to skin SK. The string-like connectors 50s make it possible to position the subsidiary patches closer to each other on the skin than they were on the release liner (or, not shown, to separate them more). FIG. 5C shows the subsidiary patches 50a and 50b removed from the release liner 50c and applied to skin SN. The string-like connectors 50s make it possible to position the subsidiary patches at an angle to each other on the skin. Optionally, one, some, or all of the subunit patches 50d, 50e may have a hole 50t through which treatment material is introducible into the sub-unit patch. This can be done before release from the release liner, after application to skin, or both.

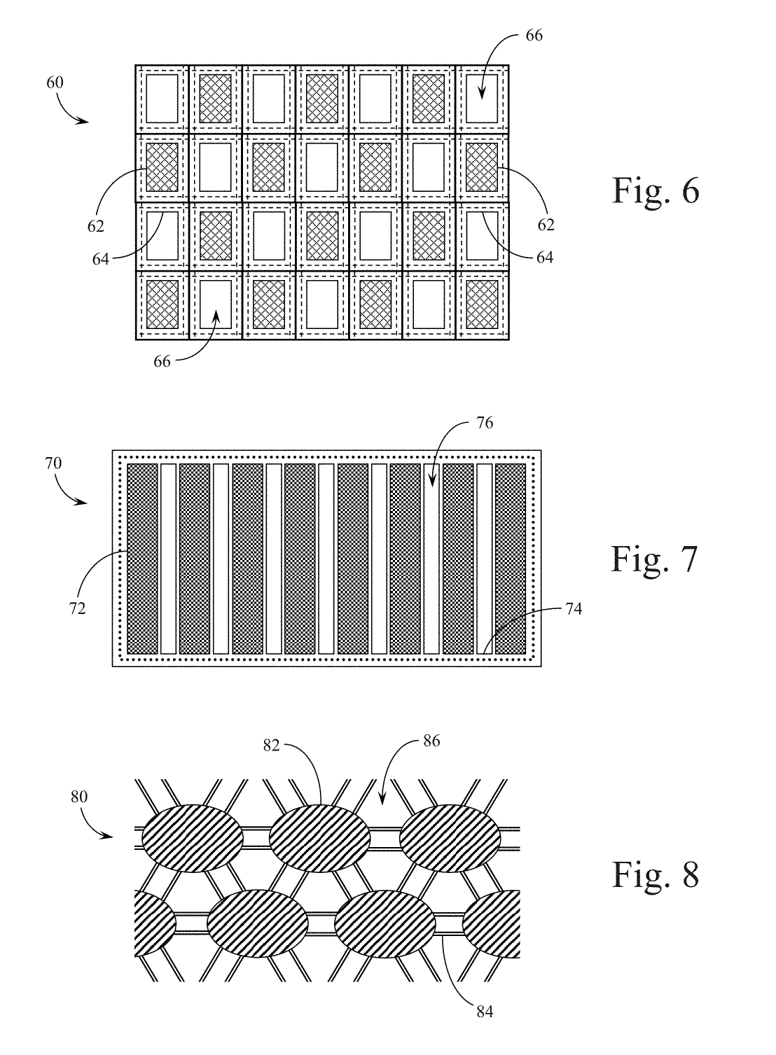

FIG. 6 shows a main patch 60 according to the present invention with subunit patches 62 interconnected by members of a connector grid 64. The subunit patches 62 may be any subunit patch disclosed herein (as is true for any subunit indicated for the embodiments of FIGS. 5-11). Perforated lines on the grid members 64 indicate that any part of the main patch 62, with any desired number of subunit patches, may be separated from the main patch and used on skin. The grid members define openings 66 through the patch structure.

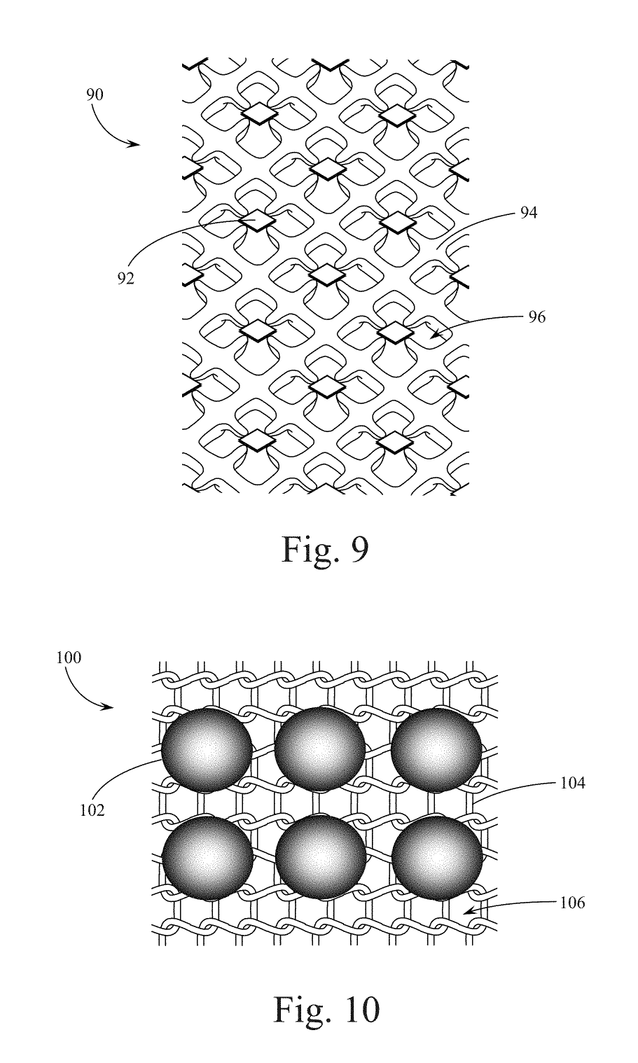

FIG. 7 shows a main patch 70 according to the present invention with subunit patches 72 interconnected by members of a connector frame 74. The subunit patches 72 may be any subunit patch disclosed herein. Portions of the frame 74 and of the subunit patches 72 define openings 76 through the patch structure. FIG. 8 shows a main patch 80 according to the present invention with subunit patches 82 interconnected by connectors 84. The subunit patches 82 may be any subunit patch disclosed herein. Portions of the connectors 84 and of the subunit patches 82 define openings 86 through the patch structure. FIG. 9 shows a main patch 90 with subunit patches 92 interconnected by parts of a connector network 94. Portions of the connector network 94 and of the subunit patches 92 define openings 96 through the patch structure. FIG. 10 shows a main patch 100 with subunit patches 102 interconnected by a chain mail structure 104. Portions of the subunit patches and of the chain mail structure 104 define openings 106 through the patch structure.

It is within the present invention, as desired, to eliminate from any embodiment the openings through a main patch structure. That is, for certain embodiments there are no openings and openings are not required, nor their functions, for a working, efficient patch. Such a patch without openings may still have connectors between subunit patches, or not.

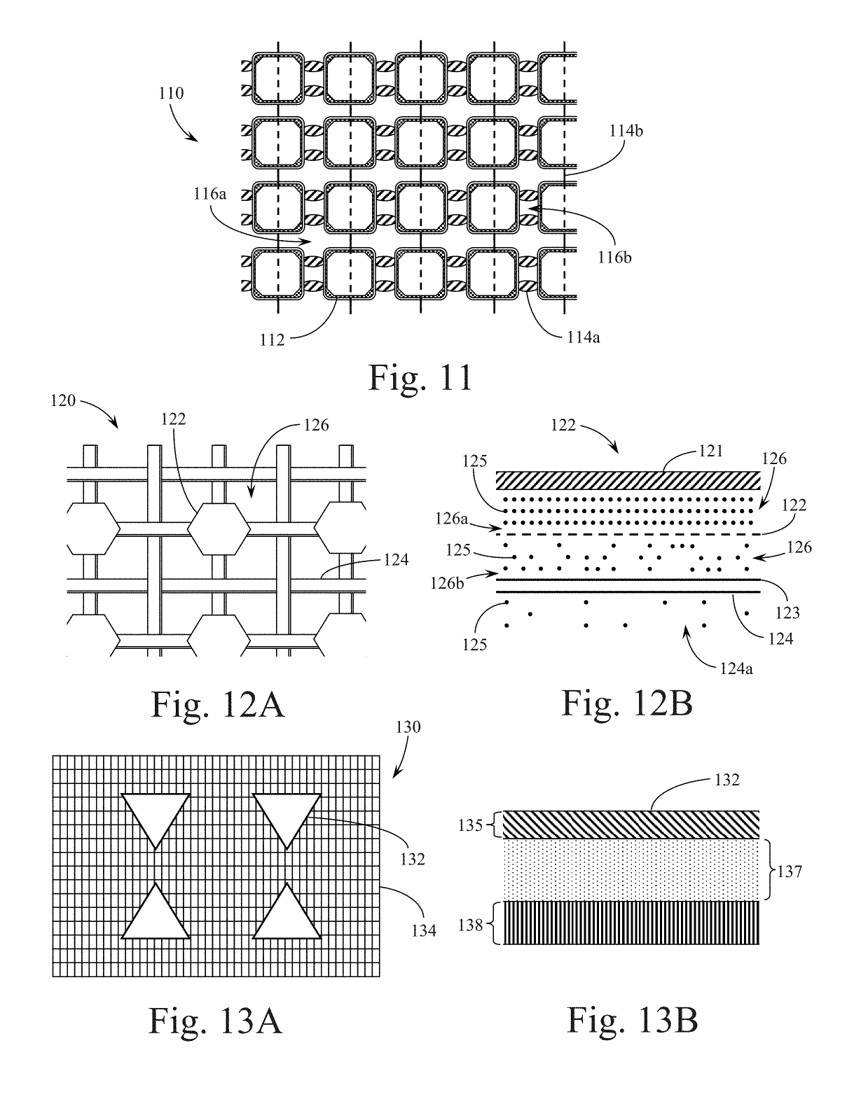

It is within the scope of the present invention to provide a main patch with multiple subunit patches each of which is interconnected with adjacent patches by a plurality of connectors, the subunit patches being a patch such as any patch disclosed in U.S. Pat. No. 9,326,952, the disclosure of which is incorporated herein by reference in its entirety for all purposes. Such a main patch is new and nonobvious, has new and nonobvious structure, new and nonobvious functions, and achieves new and nonobvious results as compared to the disclosure of U.S. Pat. No. 9,326,952, One such main patch is shown in FIG. 11, Any main patch disclosed herein may have one or some or all its subunit patches like those disclosed in U.S. Pat. No. 9,326,952. FIG. 11 shows a main patch 110 according to the present invention which has a plurality of subunit patches 112 connected to adjacent subunit patches with connectors 114a and 114b. It is to be understood that any desired number of subunit patches may be used, and any connector or connectors disclosed herein may be used to interconnect the subunit patches. Surfaces of the subunit patches and of the connectors define open spaces 116a and 116b between the subunit patches. In certain aspects, the subunit patches 112 are any of the patches disclosed in U.S. Pat. No. 9,326,952, which include a support, an adhesive layer containing a treatment material, including but not limited to a drug or medicine, and arranged on at least one side of the support, and a release liner arranged on a side of the adhesive layer opposite to the side thereof on which the support is arranged, wherein the support, the release liner and the adhesive layer each have any desired shape, including but not limited to a rectangular planar shape, wherein, optionally, at one or more corners of the adhesive patch, the adhesive patch has a protrudent part on a support-side surface thereof. Such a subunit patch may be provided in any form of a matrix-type or reservoir-type.

Any suitable support may be used for each subunit patch 112. One such support usable in the subunit patches 112 of the invention includes a single film of polyester resins such as polyethylene terephthalate, etc.; polyamide resins such as nylon, etc.; olefinic resins such as polyethylene, polypropylene, etc.; vinylic resins such as ethylene-vinyl acetate copolymer, polyvinyl chloride, polyvinylidene chloride, ionomer resin, etc.; acrylic resins such as ethylene-ethyl acrylate copolymer, etc.; fluorocarbon resins such as polytetrafluoroethylene, etc.; metal foil or the like, and/or a laminate film of these. The thickness of the support can be generally from 10 μm to 200 μm, preferably from 15 μm to 150 μm, more preferably from 20 μm to 100 μm.

For enhancing the adhesiveness (anchor-ability) between the support and the adhesive layer, the support (and any support of any embodiment disclosed herein) can be a nonporous laminate film formed of the above-mentioned material and a porous film, in which the adhesive layer is formed on the side of the porous film. Not specifically defined, the porous film may be any one capable of enhancing the anchor-ability between the support and the adhesive layer, including, for example, paper, woven fabric, nonwoven fabric, mechanically-perforated film, etc. The thickness of the porous film can be any desired thickness, for example, from 10 um to 100 um in consideration of improving anchor-ability and the flexibility of the adhesive layer. One such support is a laminate film of a polyester-based resin film (for example, a polyethylene terephthalate film) having a thickness of from 1 μm to 45 μm and a polyester-based resin (for example, polyethylene terephthalate) formed nonwoven fabric having a thickness of from 10 μm to 100 μM.

The adhesive that constitutes the adhesive layer in the adhesive patch of the invention may include an acrylic adhesive including an acrylic copolymer; a rubbery adhesive such as styrene-diene-styrene block copolymer (for example, styrene-isoprene-styrene block copolymer, styrene-butadiene-styrene block copolymer, etc.), polyisoprene, polyisobutylene, polybutadiene, etc.; a silicone-based adhesive such as silicone rubber, dimethylsiloxane base, diphenylsiloxane base, etc.; a vinyl ether-based adhesive such as polyvinyl methyl ether, polyvinyl ethyl ether, polyvinyl isobutyl ether, etc.; a vinyl ester-based adhesive such as vinyl acetate-ethylene copolymer, etc.; a polyester-based adhesive including a carboxylic acid component such as dimethyl terephthalate, dimethyl isophthalate, dimethyl phthalate or the like, and a polyalcohol component such as ethylene glycol or the like, etc. The adhesive layer formed of the adhesive may be crosslinked through physical crosslinking treatment of exposure to radiation such as UV irradiation, electron beam irradiation or the like, or through chemical crosslinking treatment with various crosslinking agents, or may also be a non-crosslinked adhesive layer with no crosslinking treatment applied thereto. The adhesive layer may contain a tackifier.

The drug or medicine on or contained in the adhesive layer may be one of: any systemic drugs or local-acting drug; any transdermal drugs that are administrable to mammals including humans via the skin thereof; and, for example, drugs that include systemic anesthetics, antipsychotics, antidepressants, mood stabilizers, psychostimulants, narcotics, anxiolytics, antiepileptic drugs, Migraine medications, antiemetics, anti-vertiginous drugs, local anesthetics, muscle relaxants, autonomic agents, antispasmodics, Parkinson disease remedies, corticosteroids, pain killers, mepivacaine, nonsteroidal anti-inflammatory drugs, analgesic-antipyretics, anti-rheumatic drugs, antihistamines, anti-allergies, cardiotonics, antiarrhythmics, diuretics, anti-hypertensives, vasoconstrictors, vasodilators, angina remedies, respiratory stimulants, bronchodilators, bronchial asthma remedies, antitussives, expectorants, hormone preparations, hematinics, hemostats, antithrombotic drugs, gout hyperuricemic remedies, diabetes remedies, hypolipidemic drugs, antineoplastics, immune-suppressants, antimicrobials, chemotherapeutics, antifungals, antivirals, antiparasitics, narcotics, stop smoking aids, etc. The subunit patches of this invention may contain the drug as a free form or as a salt with an acid or a base. The salt of the drug includes alkali metal salts such as sodium salts, potassium salts, etc.; alkaline earth metal salts such as magnesium salts, calcium salts, etc.; inorganic acid salts such as hydrochlorides, nitrates, sulfates, etc.; organic acid salts such as acetates, citrates, fumarates, maleates, etc.; salts with inorganic bases such as ammonium salts, etc.; salts with organic bases such as triethanolamine salts, pyridine salts, arginine salts, etc.

The release liner of the subunit patches 112 may include glassine paper, polyethylene, polypropylene, polyesters such as polyethylene terephthalate, etc., polystyrene, aluminum film, foamed polyethylene film, foamed polypropylene film, etc.; as well as laminates selected from these, silicone-processed or embossed products of these, etc. The thickness of the release liner may be generally from 10 μM to 200 μm, for example from 25 μm to 100 μm. There are two connectors 114a between adjacent subunit patches 112, but any desired number of such connectors may be used, of any desired shape. There is one connector 114b between adjacent patches, but any desired number of such connectors may be used. Some or all of the connectors 114a may be replaced by connectors 114b, and vice versa. In certain aspects, the connectors 114b are string-like connectors. The support, adhesive layer, and/or release liner of the patch 110 may be used in any patch according to the present invention that has one or more of such parts.

It is within the scope of the present invention to provide a mainpatch with multiple subunit patches each of which is interconnected with adjacent patches by a plurality of connectors, the subunit patches being a patch such as any patch disclosed in U.S. Pat. No. 5,683,712, the disclosure of which is incorporated fully herein in its entirety for all purposes. Such a main patch is new and nonobvious as compared to the patches disclosed in U.S. Pat. No. 5,683,712; and such a main patch has new and nonobvious structure, new and nonobvious functions, and achieves new and nonobvious results as compared to the subject matter of U.S. Pat. No. 5,683,712. One such main patch is shown in FIG. 12A. Any main patch disclosed herein may have one or some or all its subunit patches like those of U.S. Pat. No. 5,683,712.

FIG. 12A shows a main patch 120 according to the present invention which has a plurality of subunit patches connected to adjacent subunit patches with connectors which are strands of a woven material 124. It is to be understood that any desired number of subunit patches may be used and any suitable woven material, natural or synthetic, may be used tor the material 124, Surfaces of the subunit patches and of the woven material define open spaces 126 between the subunit patches. In certain aspects, the subunit patches 122 are any of the patches disclosed in U.S. Pat. No. 5,683,712, which include structures as illustrated in FIG. 12B. Such subunit patches include a support membrane, a layer of porous adhesive, a microporous membrane and a substance, tor example, but not limited to, a gel, containing treatment material to be administered. The microporous membrane can be a reservoir for the treatment material. In certain such patches, when in use on a person's or an animal's skin, as shown in FIG. 12B, the subunit patch presents a sandwich-type structure with support membrane 121 on the outer surface of the patch distal to the patient's skin and a layer of porous adhesive (for example, silicone) 123 on the opposite side which defines the contact surface of the patch with the epidermis 124 of the area of the body chosen for the application.

Between support membrane 121 and porous adhesive layer 123 there is a microporous membrane 122 which controls the release of treatment material 125, for example, but not limited to a drug, essential oil, or medicine. Material 125 is scattered. in a substance, for example, but not limited to, a mass of porous material or a gel, for example, but not limited to a gel of glycerine, distilled water, lactose, polyvinyl alcohol, polyvinyl-2-pyrrolidone, and sodium citrate. Zone 126a in FIG. 12B, situated between support membrane 121 and microporous membrane 122 acts as a treatment material reservoir so that the concentration of material in this zone is maximal, whereas, because of the effect of membrane 122, the concentration in zone 126b below the membrane is lower, while the concentration in the subcutaneous zone 124a is even lower owing to the regulatory effect which the skin itself exerts on the spread of the material. In FIG. 12B, the progressively decreasing concentration in zones 126a, 126b and 124a, respectively, is represented schematically by a correspondingly decreasing density of dots denoting molecules of material. Layers, connectors and membranes of the patch 120 (see FIG. 12A) may be used, as desired and as appropriate, in any other embodiment of the present invention.

It is within the scope of the present invention to provide a main patch with multiple subunit patches each of which is interconnected with adjacent patches by a plurality of connectors, the subunit patches being a patch such as any patch disclosed in U.S. Patent Application Publication No. 2016/0374956, the disclosure of which is incorporated fully herein in its entirety for all purposes. Such a main patch is new and nonobvious as compared to the patches disclosed in U.S. Patent Application Publication No. 2016/0374956; and such a main patch has new and nonobvious structure, new and nonobvious functions, and achieves new and nonobvious results as compared to the subject matter of U.S. Patent Application Publication No. 2016/0374956. One such main patch is shown in FIG. 13A. Any main patch disclosed herein may have one or some or all its subunit patches like those of U.S. Patent Application Publication No. 2016/0374956. FIG. 13A shows a main patch 130 according to the present invention which has a plurality of subunit patches 132 connected to adjacent subunit patches with connectors which are strands of a mesh 134. The subunit patches may be within the mesh, that is, the mesh material may not extend under or over the patch surfaces, or the subunit patches may be on the mesh itself. Each subunit patch can include a support layer (see layer 135, FIG. 13B); a treatment material reservoir layer (layer 137, FIG. 13B); and an adhesive layer (layer 138, FIG. 139); and optionally a release liner (not shown, beneath the adhesive layer).

In certain aspects, such a subunit patch as subunit patch 132, has as a support any support layer that can support the treatment material reservoir layer. Any known support layer for a patch can be employed, as appropriate, as the support layer according to the present invention, Examples of the material of the support layer (and Which can be the material of any support layer or backing herein) include, but are not limited to, synthetic resins such as polyesters including polyethylene terephthalate, polybutylene terephthalate, and polyethylene naphthalate; polyolefins such as polyethylene and polypropylene; polyurethanes; and ethylene-vinyl acetate copolymer, as well as metals such as aluminum, and paper. In addition, the form of the support layer made of such a material is, for example, a film; a sheet such as a foamed sheet, a porous sheet, or a microporous sheet; a fabric such as a woven fabric, a knitted fabric, or a nonwoven fabric; a foil; or a laminate of any of these. The support layer according to the present invention may be impermeable to the treatment material. In one aspect, the support layer is a polyester film which has flexibility and impermeability. In addition, a thickness of the support layer is not particularly limited, either, and can be, for example, about 2 μm to 600 μm.

The treatment material used with the subunit patches may include, as is true for any patch or subunit patch according to the present invention, within a range not impairing an effect of the present invention: hypnotic and sedative drugs (flurazepam hydrochloride, rilmazafone hydrochloride, phenobarbital, amobarbital, and the like), antipyretic and anti-inflammatory agents (butorphanol tartrate, perisoxal citrate, acetaminophen, mefenamic acid, diclofenac sodium, aspirin, alclofenac, ketoprofen, flurbiprofen, naproxen, piroxicam, pentazocine, indomethacin, glycol salicylate, aminopyrine, loxoprofen, and the like), steroidal anti-inflammatory agents (hydrocortisone, prednisolone, dexamethasone, betamethasone, and the like), analeptics and stimulants (methamphetamine hydrochloride, methylphenidate hydrochloride, and the like), neuropsychiatric agents (imipramine hydrochloride, diazepam, sertraline hydrochloride, fluvoxamine maleate, paroxetine hydrochloride, citalopram hydrobromide, fluoxetine hydrochloride, alprazolam, haloperidol, clomipramine, amitriptyline, desipramine, amoxapine, maprotiline, mianserin, setiptiline, trazodone, lofepramine, milnacipran, duloxetine, venlafaxine, chlorpromazine hydrochloride, thioridazine, diazepam, meprobamate, etizolam, risperidone, mirtazapine, and the like), hormone drugs (estradiol, estriol, progesterone, norethisterone acetate, methenolone acetate, testosterone, and the like), local anesthetics (lidocaine hydrochloride, procaine hydrochloride, tetracaine hydrochloride, dibucaine hydrochloride, propitocaine hydrochloride, and the like), agents for urinary organs (oxybutynin hydrochloride, tamsulosin hydrochloride, propiverine hydrochloride, tolterodine tartrate, imidafenacin, and the like), skeletal muscle relaxants (tizanidine hydrochloride, eperisone hydrochloride, pridinol mesylate, suxamethonium chloride, and the like), agents for reproductive organs (ritodrine hydrochloride and meluadrine tartrate), perfumes, colognes, odor-masking material; antiepileptic agents (sodium valproate, clonazepam, carbarnazepine, and the like), autonomic agents (carpronium chloride, neostigmine bromide, bethanechol chloride, and the like), antiparkinsonian agents (pergolide mesylate, bromocriptine mesylate, trihexyphenidyl. hydrochloride, amantadine hydrochloride, ropinirole hydrochloride, talipexole hydrochloride, cabergoline, droxidopa, biperiden, selegiline hydrochloride, and the like), diuretic agents (hydroflumethiazide, furosemide, and the like), respiratory stimulants (lobeline hydrochloride, dimorpholamine, naloxone hydrochloride, and the like), antimigraine agents (dihydroergotamine mesylate, sumatriptan, ergotamine tartrate, flunarizine hydrochloride, cyproheptadine hydrochloride, and the like), antihistamines (clemastine fumarate, diphenhydramine tannate, chlorphenirarn maleate, diphenylpyraline hydrochloride, promethazine, and the like), bronchodilators (tulobuterol hydrochloride, procaterol hydrochloride, salbutamol sulfate, clenbuterol hydrochloride, fenoterol hydrobromide, terbutaline sulfate, isoprenaline sulfate, formoterol fumarate, and the like), cardiotonics (isoprenaline hydrochloride, dopamine hydrochloride, and the like), coronary vasodilators (diltiazem hydrochloride, verapamil hydrochloride, isosorbide mononitrate, nitroglycerin, nicorandil, and the like), peripheral vasodilators (nicametate citrate, tolazoline hydrochloride, and the like), smoking cessation aids (nicotine, and the like), agents for circulatory organs (flunarizine hydrochloride, nicardipine hydrochloride, nitrendipine, nisoldipine, felodipine, amlodipine besylate, nifedipine, nitvadipine, manidipine hydrochloride, benidipine hydrochloride, enalapril maleate, temocapril hydrochloride, alacepril, imidapril hydrochloride, cilazapril, lisinopril, captopril, trandolapril, perindopril erburnine, atenolol, pindolol, bisoprolol fumarate, metoprolol tartrate, betaxolol hydrochloride, timolol maleate, bopindotol malonate, nipradilol, arotinolol hydrochloride, celiprolol hydrochloride, carvedilol, amosulalol hydrochloride, carteolol hydrochloride, bevantolol hydrochloride, terazosin hydrochloride, bunazosin hydrochloride, prazosin hydrochloride, doxazosin mesylate, valsartan, candesartan cilexetil, losartan potassium, clonidine hydrochloride, guanfacine hydrochloride, guanabenz acetate, and the like), antiarrhythmic agents (propranolol hydrochloride, alprenolol hydrochloride, procainamide hydrochloride, mexiletine hydrochloride, nadolol, disopyramide, and the like), anti-malignant-ulcer agents (cyclophosphamide, fluorouracil, tegafur, procarbazine hydrochloride, ranimustine, irinotecan hydrochloride, fluridine, and the like), antilipemic agents (pravastatin, simvastatin, bezafibrate, probucol, and the like), hypoglycemic agents (glibenclamide, chlorpropamide, tolbutamide, glymidine sodium, glybuzole, buforinin hydrochloride, and the like), anti-peptic ulcer agents (proglumide, cetraxate hydrochloride, spizofurone, cimetidine, glycopyrronium bromide, and the like), cholagogues (ursodesoxycholic acid, osalmid, and the like), gastroprokinetic agents (domperidone, cisapride, and the like), agents for hepatic diseases (tiopronin and the like), anti-allergic agents (ketotifen fumalate, azelastine hydrochloride and the like), antiviral agents (acyclovir and the like), antivertigo agents (betahistine mesylate, difenidol hydrochloride, and the like), antibiotics (cephaloridine, cefdinir, cefpodoxime proxetil, cefaclor, clarithromycin, erythromycin, methyl ethromycin, kanamycin sulfate, cycloserine, tetracycline, benzylpenicillin potassium, propicillin potassium, cloxacillin sodium, ampicillin sodium, bacampicillin hydrochloride, carbenicillin sodium, chloramphenicol, and the like), agents for habitual intoxication (cyanamide and the like), appetite suppressants (mazindol and the like), chemotherapeutic agents (isoniazid, ethionamide, pyrazinamide, and the like), blood coagulation accelerators (ticlopidine hydrochloride, warfarin potassium, and the like), anti-Alzheimer's agents (physostigmine, donepezil hydrochloride, tacrine, arecoline, xanomeline, and the like), serotonin receptor antagonist antiemetics (ondansetron hydrochloride, granisetron hydrochloride, ramosetron hydrochloride, azasetron hydrochloride, palonosetron, and the like), antigout agents (colchicine, probenecid, sulfinpyrazone, and the like), narcotic analgesics (fentanyl citrate, morphine sulfate, morphine hydrochloride, codeine phosphate, cocaine hydrochloride, pethidine hydrochloride, and the like).

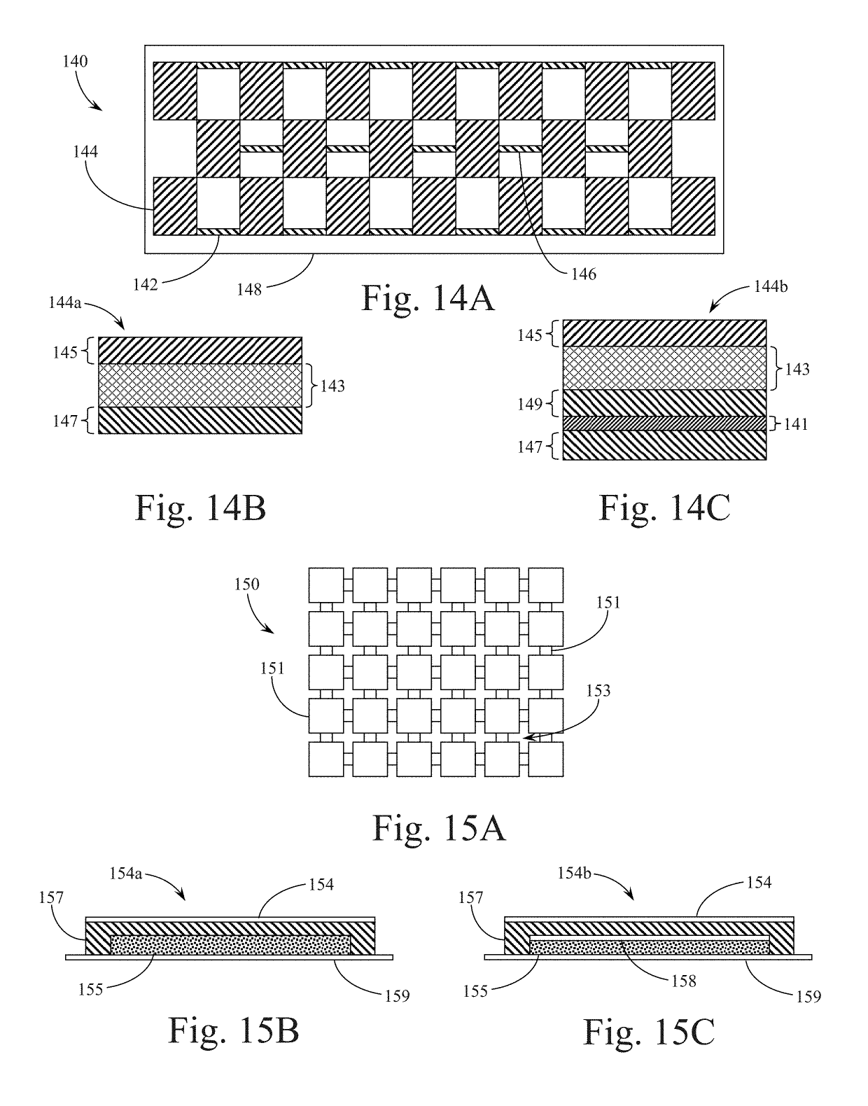

It is within the scope of the present invention to provide a main patch with multiple subunit patches each of Which is interconnected with adjacent patches by a plurality of connectors, the subunit patches being a patch such as any patch disclosed in U.S. Pat. No. 8,956,644, the disclosure of which is incorporated fully herein in its entirety for all purposes. Such a main patch is new and nonobvious as compared to the patches disclosed in U.S. Pat. No. 8,956,644; and such a main patch has new and nonobvious structure, new and nonobvious functions, and achieves new and nonobvious results as compared to any subject matter of U.S. Pat. No. 8,956,644. One such main patch is shown in FIG. 14A. Any main patch disclosed herein may have one or some or all its subunit patches like any of those of U.S. Pat. No. 8,956,644. A main patch 140 has a plurality of subunit patches 144 connected by connectors 142 and 146. Initially, the main patch 140 may be positioned on and releasably adhered to a release layer 148. The subunit patches 144 may, in certain aspects, be like any suitable subunit patch disclosed herein. In certain aspects, the subunit patches are like one of the patches disclosed in U.S. Pat. No. 8,956,644, of suitable size, shape, and configuration for use in a main patch according to the present invention. Such a subunit patch may be a system with a backing layer; a reservoir; and a release liner; wherein the reservoir is an adhesive type matrix with treatment material or into which treatment material is introducible. Any treatment material may be used, including but not limited to an anesthetic, including but not limited to bupivacaine.

In certain aspects, such a subunit patch has a backing layer; a reservoir connected to the backing layer, for example, but not limited to, by lamination; and a release liner; wherein the reservoir is a liquid reservoir, a gel reservoir, or a matrix reservoir; and, in one particular aspect is an adhesive type matrix including a blend of: (i) a high molecular weight polyisobutylene having a viscosity average molecular weight of 450,000 to 2,100,000; and (ii) a low molecular weight polyisobutylene having a viscosity average molecular weight of 1,000 to 450,000; and the reservoir includes sucrose acetate isobutyrate in an amount effective to reduce a peel force of the system; and the reservoir includes bupivacaine In certain of these subunit patches the reservoir includes a plasticizer, which may be one of polybutene, mineral oil, linseed oil, octyl palmitate, squalene, silicone oil, isobutyl stearate, olive oil, isopropyl myristate, isostearyl alcohol and oleyl alcohol. In certain of these subunit patches, the backing layer includes or is a breathable r a of woven or non-woven fabric, and, in one aspect is polyester. The reservoir may have a thickness between 1 to 50 mils, and in other aspects, between 1 to 10 mils.

Subunit patches like the subunit patches 144, can include a backing layer that provides a protective outer surface for the patches, and, optionally, a release liner or layer that will cover the adhesive portion of the device that is used to affix the same to the skin of a subject. The release liner is removed prior to application, thereby exposing the adhesive portion of the device, which will typically be a pressure-sensitive adhesive. Accordingly, referring to FIGS. 14B and 14C which show exemplary embodiments of subunit patches 144a and 144b, respectively, for a dermal or transdermal delivery system. The subunit patch includes a backing layer 145, a reservoir 143 that contains treatment material (including, but not limited to, bupivacaine), and a release liner 147 (removed before application to skin). The reservoir may be a liquid or gel reservoir, or it may be a matrix carrier that can be self-adhesive or non-adhesive. Referring to FIG. 15C, in those patches where the reservoir is either a liquid or gel reservoir, or a non-adhesive matrix, the patch also has an adhesive layer 141 that serves to adhere the device to the skin. The adhesive layer may, in certain aspects, be generally a drug-permeable, drug-compatible, and inert adhesive that is applied over the reservoir. Optionally a layer 149 is used as a rate controlling membrane, where the layer is selected to provide for selective movement of treatment material, for example, but not limited to, bupivacaine, through the layer.

The backing layer 145, which adheres to the treatment-material-containing reservoir, serves as the upper layer of the device during use and functions as the primary structural element of the device. The backing layer is thus typically a sheet or film of a preferably flexible elastomeric material. This backing layer 145 may have a thickness of about 0.1 to 50 mils, preferably about 0.5 to 30 mils, and more preferably about 1 to 25 mils (as is true for any backing layer or top or top cover disclosed herein). The backing layer may further be a monolithic (single layer) or a multi-layer (multi-laminate), as is true for any backing layer, top or top cover of any embodiment herein), and may further be a breathable or occlusive material of woven or non-woven fabric. The backing layer may be a polymeric material, or a laminate of polymeric materials. Suitable materials include, but are not limited to, polyethylene, polypropylene, polyesters, a nonwoven polyester fabric, polyurethanes, polyethylene vinyl acetate, polyvinylidene chloride, block copolymers such as Pebax®, polyvinyl acetate, polyvinylidene chloride, polyurethane, ethylene vinyl acetate, polyethylene terephthalate, polybutylene terephthalate, coated paper products, metal or metalized sheets and the like, and any combinations thereof.

The reservoir 143 (as is true for any reservoir herein or any treatment material layer or layer with treatment material) is disposed on the backing layer. The reservoir may be formed from any number of standard materials. In those subunit patches where the reservoir is a liquid or get-type reservoir, any suitable gelling agent may be used to form an aqueous gel system, for example cellulose materials. In those devices where the reservoir is a matrix-type reservoir, it may be formed from any polymeric material in which the treatment material is soluble, for example, but not limited to bupivacaine which has some solubility within a desired solubility range, for example, a polyurethane, ethylenelvinyl acetate copolymer (EVA), polyacrylate, styrenic block copolymer, and the like.

The reservoir may be, or may include, an adhesive type matrix, formed from a pharmaceutically acceptable pressure sensitive adhesive, for example a polyisobutylene, polyacrylate or a styrenic block copolymer-based adhesive. In certain aspects in which the patch is provided as a monolithic, adhesive matrix device, the reservoir can be formed from standard pressure sensitive adhesives known in the art. Suitable pressure sensitive adhesives for use in the practice of the invention thus include, but are not limited to, polyacrylates, polysiloxanes, polyisobutylene polyisoprene, polybutadiene, styrenic block polymers, blends and combinations of the above, and the like. Suitable styrenic block copolymer-based adhesives include, but are not limited to, styrene-isoprene-styrene block copolymer (SIS), styrene-butadiene-styrene copolymer (SBS), styrene-ethylene-butylene-styrene copolymers (SEBS), and di-block analogs thereof. Suitable acrylic polymers are comprised of a copolymer or terpolymer comprising at least two or more exemplary components selected from acrylic acids, alkyl acrylates, methacrylates, co-polymerizable secondary monomers or monomers with functional groups. Examples of monomers include, but are not limited to, acrylic acid, methacrylic acid, methoxyethyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethyihexyl methacrylate, decyl acrylate, decyl methacrylate, dodecyl acrylate, dodecyl methacrylate, tridecyl acrylate, tridecyl methacrylate, hydroxyethyl acrylate, hydroxypropylacrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, rnethoxyethyl acrylate, methoxyethyl methacrylate, and the like. In a preferred embodiment, the pressure-sensitive adhesive is an acrylate having no functional groups or cross linkers (for example, DURO-TAK® 87-9301, available from National Starch & Chemical, Bridgewater, N.J.), or a blend of acrylate-vinyl-acetates having —COOH and —OH functional groups (DURO-TAK® 87-2051 and 87-2287, National Starch & Chemical).

In certain other embodiments, the reservoir is formed from a monolithic adhesive matrix containing a polyisobutylene material. The polyisobutylene may comprise a blend of a high molecular weight polyisobutylene (about 450,000 to 2,100,000 viscosity average molecular weight) and a low molecular weight polyisobutylene (about 1,000 to 450,000 viscosity average molecular weight). The reservoir, with or without an adhesive coating, may have a thickness ranging from about 1 to about 10 mils. The reservoir (and any of any embodiment herein) may include optional ingredients, such as carriers, vehicles, additives, excipients, stabilizers, dyes, diluents, plasticizers, tackifying agents, crystallization inhibitors, solubility enhancers, inert fillers, antioxidants, anti-irritants, vasoconstrictors and other materials without pharmacological activity that are suitable for administration in conjunction with the transdermal delivery systems of the present invention. In certain aspects, the reservoir may include one or more rheology and/or surface energy modifying agents (“RSEMA”) that improve the adhesive properties of the device, for example by promoting skin friendly removal and reapplication of the present transdermal delivery systems. Optionally, the reservoir can include one or more filler materials. Suitable fillers include, but are not limited to, metal oxides, inorganic salts, synthetic polymers, clays and the like. The metal oxides may be silicon dioxide, zinc oxide, magnesium oxide, titanium oxide, and calcium oxide. Inorganic salts can be calcium, magnesium and sodium carbonate, calcium and magnesium sulfate, calcium phosphate, and the like. Synthetic polymers can include methacrylic resin, nylon, polyethylene, and the like. Suitable clay compounds include talc, bentonite and kaolin.

The release liner may be a peelable layer which is a disposable element that serves to protect the patch prior to application to the skin. The release liner may be formed from a material impermeable to the treatment material (as is true for any release liner or layer herein) and other components of the patch, and easily removable from the reservoir. Suitable materials include various polymeric materials that may be optionally metallized. Examples of suitable polymeric materials comprise polyurethane, polyvinyl acetate, polyvinylidene chloride, polypropylene, polycarbonate, polystyrene, polyethylene, polyethylene terephthalate, polybutylene terephthalate, polymer coated papers and combinations thereof In preferred embodiments, the protective layer comprises a siliconized sheet (for example Medirelease® Paper Silicone or PE from Mylan Tech, St. Albans, Vt.), or has a fluoropolymer coating (for example Scotchpak™ 9744 (available from 3M, St. Paul, Minn.).

Subunit patches of the present invention, including but not limited to those of the main patch 140, can be manufactured. for example, to produce matrix-type patches, beginning with a solution of a suitable polymeric reservoir material added to a double planetary mixer, followed by addition of desired amounts of treatment material. Typically, the polymeric reservoir material is an adhesive polymer, which can be solubilized in an organic solvent, for example, ethanol, ethyl acetate, and hexane. After mixing has taken place for a suitable period of time to achieve acceptable uniformity of the ingredients, the resultant mixture can be feel into a casting die. In such cases, the matrix mixture is cast as a wet film onto a release liner carried on a moving web or belt, which is drawn through lines. A series of ovens is then used to evaporate the casting solvent to acceptable residual limits. A selected backing membrane can then be laminated onto the dried reservoir film. In subsequent operations, individual patches are separated and unit-packaged. In other processes, a reservoir can be formed using dry-blending and thermal film-forming. The materials are dry blended and extruded using a slot die followed by calendering to an appropriate thickness.

It is within the scope of the present invention to provide a main patch with multiple subunit patches each of which is interconnected with adjacent patches by a plurality of connectors, the subunit patches being a patch, of appropriate size according to the present invention, such as any patch disclosed in U.S. Pat. No. 9,656,441 which is incorporated fully herein in its entirety for all purposes. Such a main patch is new and nonobvious as compared to the patches disclosed in U.S. Pat. No. 9,656,441; and such a main patch has new and nonobvious structure, new and nonobvious functions, and achieves new and nonobvious results as compared to the subject matter of U.S. Pat. No. 9,656,441. One such main patch is shown in FIG. 15A. Any main patch disclosed herein may have one or some or all of its subunit patches like any of those of U.S. Pat. No. 9,656,441.

In certain aspects, a main patch according to the present invention has a plurality of interconnected subunit patches, each of which has a protective layer, a matrix layer, an adhesive layer, and a cover layer. In certain aspects, the cover layer may be at least partially bi-elastic, and the adhesive layer may include an acrylic copolymer having hydroxyl functional groups. The acrylic copolymer having hydroxyl functional groups may be crosslinked. The acrylic copolymer may comprise a combination of 2-ethylhexyl acrylate, glycidyl methacrylate, 2-hydroxyethyl acrylate, and vinyl acetate. The acrylic copolymer may also comprise a combination of 2-ethylhexyl acrylate, 2-hydroxyethyl acrylate, and vinyl acetate. The matrix layer may have or have introduced to it any desired treatment material, and, in certain aspects, has a physiologically effective amount of buprenorphine or pharmaceutically acceptable salts thereof.

In certain aspects, the matrix layer may be arranged so as to be in contact with the protective layer and in contact with the adhesive layer, but not in contact with the cover layer. The adhesive layer may be arranged to be in contact with the cover layer and may be optionally in contact with the protective layer. A separating layer is optionally between the matrix layer and the adhesive layer. The cover layer may have an elasticity in the longitudinal and transverse direction of 20% or more. In certain aspects, the adhesive layer does not contain an active drug, such as buprenorphine.

In certain embodiments, the present invention is directed to processes for preparing a subunit patch, which comprises: providing a treatment material or a treatment material composition and/or combination, which, in one aspect is a pain killer, and in one particular aspect is an active drug composition, which contains an active drug, for example buprenorphine, and producing matrix layer therefrom, which contains the treatment material and/or active drug, providing a composition for an adhesive layer, for example but not limited to a composition comprising an acrylic copolymer having hydroxyl functional groups for producing an adhesive layer, providing a protective layer, providing a bi-elastic cover layer, and combining the above listed components in the layer order described above to produce a transdermal patch according to any suitable embodiment of the present invention.

In other aspects, the present invention provides processes for making subunit patches which include: producing a laminate comprising a matrix layer which contains the treatment material (any suitable such material disclosed herein), in one exemplary aspect a treatment material which is an active drug, such as buprenorphine and a protective layer, preparing a laminate comprising an adhesive layer, which in certain aspects may include an acrylic copolymer having hydroxyl functional groups, and a cover layer, and connecting the two laminates to produce the subunit patch. Any such subunit patch made by any of these processes may include a top surface of the matrix layer covered with a separating layer. In addition, the laminate with the adhesive layer may also have an interim protective layer on the surface of the adhesive layer, which may be removed from the laminate before connecting the laminate with the laminate comprising the matrix layer.

For any main patch according to the present invention with multiple subunit patches, the present invention provides a use of the patch and patches for the treatment, alleviation, and/or prophylaxis of pain. Also, the present invention provides embodiments in which the use of the patch and patches is directed to a method or methods according to the present invention for the treatment, alleviation, and/or prophylaxis of pain by administering a main patch and/or subunit patches according to the present invention to a patient in need thereof.

FIG. 15A shows a top view of a main patch 150 according to the present invention which has a plurality of subunit patches 151 connected together by a plurality of connectors 152, with open areas 153 between subunit patches. As shown in certain embodiments for the subunit patches in FIGS. 15B and 15C, the subunit patches may have a cover layer 154 on a side that does not contact skin. In certain aspects, this cover layer is flexible or stretchable and, in one aspect, is made of an elastic or of a bi-elastic. A protective layer 159 is on the side of the subunit patch opposite the side of the cover layer. A matrix layer 155, which contains treatment material, tor example but not limited to a drug or drugs, is on top of the protective layer 159. An adhesive layer 156 is present on the sides of and above the matrix layer 155 (but it is within the scope of the present invention for the adhesive layer not to be present on the sides of the matrix lays. The adhesive layer is between the cover layer and the protective layer.

In certain aspects, any connector according to the present invention, and any connector of the main patch 150, in a stable unstretched condition has elasticity or stretch ability in the longitudinal direction (lengthwise) of more than 10%, or of more than 20%, or of more than 30%; and has longitudinal extensibility which is one of greater than or equal to 25%, more than 30% to 70%, or between 40% to 60%. The extensibility in the transverse direction is similar.

Optionally, a separating layer 158 (FIG. 15C) is provided between the matrix layer and the adhesive layer. This separating layer may be made of any suitable material, including, but not limited to, polyester (for example, PET). Commercially available products to use as the separating layer include Hostaphan® RN DMF 23 (manufactured by Mitsubishi Polyester Film, Wiesbaden, Germany) or Grade 0.6 CL PET (manufactured by Loparex, Hammond, Wis.). The thickness of the separating layer may he 10 to 125 microns.

In certain aspects, other than treatment material and/or other than a pain killer, for example but not limited to, a physiologically effective amount of buprenorphine, the matrix layer may include a pressure-sensitive adhesive. The pressure-sensitive adhesives may be the same ones as described above for the adhesive layer. In certain aspects, the matrix layer may, optionally, include one or more physiologically appropriate pharmaceutical excipients. The pharmaceutical excipients may be glycols, oils and fats, urea derivatives, saturated or unsaturated fatty alcohols or fatty acids having eight to eighteen carbon atoms or esters thereof, monoglycerides, diglycerides, triglycerides and/or terpenes, tackifiers, softeners, emulsifiers, co-solvents and/or stabilizers.

One process for producing subunit patches according to the present invention includes the steps of: producing a laminate comprising a matrix layer, a separating layer, and a protective layer; preparing a laminate comprising an adhesive layer, and a cover layer, optionally being at least partially bi-elastic, and optionally comprising an interim protective layer on the surface of the adhesive layer; optionally removing the interim protective layer of the laminate and connecting the two thus-produced laminates.

The present invention provides, inter alia, applicators for applying treatment material, which is some aspects are dermal or transdermal multi-patch structures with subunit patches connected with connectors, and open spaces between subunit patches. Such applicators in certain aspects, include: a plurality of patches (sometimes herein called “subunit patches”); a plurality of connectors; a plurality of open spaces between the patches and the connectors; each patch of the plurality of patches connected to adjacent patches by a connector or connectors of the plurality of connectors; each patch of the plurality of patches spaced apart from adjacent patches by connectors between the patches; the open spaces having boundaries defined by edges of the patches and edges of the connectors; and each patch for the application of treatment material to skin.

Any patch or applicator mentioned in the previous paragraph may have one or some, in any possible combination, of the following features, aspects, steps, and/or elements:

Treatment m in or on or introducible onto or into the patches; wherein each patch has a top surface area, as viewed from above, of one of: (a) between 1 cm² and 900 cm²; (b) between 900 cm² and 2000 cm²; and (c) over 2000 cm²; wherein each connector has a top surface area, as viewed from above, of one of: (a) between 1 cm² and 30 cm²; and (b) over 30 cm²; wherein each open space has a top surface area, as viewed from above, of: (a) between 1 cm² and 450 cm²; and (b) over 450 cm²; wherein the treatment material is one of or a combination of any two of or of any three of: medicine, drugs, essential oils, attractants, repellants, descenters, perfumes, sterilizers, cleansers, local anesthetics, anticonvulsants, skeletal muscle relaxers, systemic anesthetics such as but not limited to ketamine or others similar anesthetics, natural supplements, herbal products, and/or any pharmacologically active substance(s) that impart local and/or systemic effects; wherein the patches or subunit patches are made of one of or any possible combination of: woven material or of nonwoven material; the woven material including but not limited to cloths, fabrics, textiles, linen, gauze, cotton, dressing material, barrier material, synthetic fabrics, adhesive substances, latex, natural or synthetic rubber, elastic, compounds or structures with elastic properties, plastic(s), bio-synthetics, cell-based sheets, or any combination of two or three of these or more; the nonwoven material including but not limited to air laid material, felts, pads, mats, and material with components bonded, glued, cemented, fused, and/or sintered together.

Wherein the connectors are made of one of or any possible combination of: woven material or of nonwoven material; the woven material including but not limited to cloths, fabrics, textiles, linen, gauze, cotton, dressing material, barrier material, synthetic fabrics, adhesive substances, latex, natural or synthetic rubber, elastic, compounds or structures with elastic properties, plastic(s), bio-synthetics, cell-based sheets, or any combination of two or three of these or more; the nonwoven material including but not limited to air laid material, felts, pads, mats, and material with components bonded, glued, cemented, fused, and/or sintered together.

Wherein the patches have a shape, as viewed from above, or as viewed from a side in cross-section, which is one of square, circular, oval, quadrilateral, rectangular, pentagonal, hexagonal, heptagonal, octagonal, crescent, with or without a point or points, and which, in certain aspects, in a shape of one of FIG. 3 or a shape as in FIGS. 5A-13A. Wherein the connectors have a shape, as viewed from above, or as viewed from a side in cross-section, which is one of square, circular, oval, quadrilateral, rectangular, pentagonal, hexagonal, heptagonal, octagonal, crescent, with or without a point or points, and which, in certain aspects, in a shape of one of FIG. 4. Wherein the patches have a thickness of one of: (a) between 0.02 mm and 1 mm; (h) 1 mm to 2 cm; (c) 2 cm to 3 cm; and (d) over 3 cm. Wherein the connectors have a thickness of one of: (a) between 0.02 mm and 1 mm; (b) 1 mm to 2 cm; (c) 2 cm to 3 cm; and (d) over 3 cm. Wherein the patches and the connectors have the same thickness or wherein the patches have a different thickness than the thickness of the connectors.

Wherein each patch is connected to patches adjacent to it by at least one connector, by two connectors, by three connectors, by four connectors, by five connectors, by six connectors, by seven connectors, by connectors or by any desirable number of connectors, including, but not limited to any number of connectors between one and twelve. Wherein the patches have multiple sides and the number of connectors connected to each side is the same. Wherein the patches have multiple sides and the number of connectors connected to each side is different. Wherein the patches have multiple sides and each side is connected to an adjacent patch by one connector. Wherein the patches have four sides and each side is connected to an adjacent patch by one connector. Wherein the patches are rectangular as viewed from above. Wherein the patches are square as viewed from above. Wherein the patches are circular or oval as viewed from above.

Wherein the applicator has an applicator top surface area, the patches combined have a patches top surface area, the connectors combined have a connectors top surface area, and a combined surface area of the open spaces, an open spaces area, is at least 5% of the applicator top surface area. Wherein the applicator has an applicator top surface area, the patches combined have a patches top surface area, the connectors combined have a connectors top surface area, and the patches top surface area is at least 15% of the applicator top surface area. Wherein the applicator has an applicator top surface area, the patches combined have a patches top surface area, the connectors combined have a connectors top surface area, and the connectors top surface area is at most 50% of the applicator top surface area.

Wherein the patches are made of woven material or of nonwoven material; the woven material including but not limited to cloths, fabrics, textiles, linen, gauze, cotton, dressing material, barrier material, or any combination of two or three of these; the nonwoven material including but not limited to air laid material, felts, and material with components bonded, glued, cemented, or sintered together. Wherein the connectors are made of woven material or of nonwoven material. Wherein the connectors are made of string-like material, including, but not limited to fibers, fibrils, and fibrous material. Wherein the patches are made of one layer of material or of multiple layers of material, and have, in certain aspects, any or all of the layers shown in FIGS. 1B-2D or in FIG. 12B or 1B. Wherein the connectors are made of one layer of material or of multiple layers or material. Wherein the patches are made of layers of material including a top layer, and the top layer has at least one hole or a plurality of holes therethrough through which treatment material may be introduced into the patch.

Wherein the patches and connectors are arranged in an array, mesh, pattern, net, screen, network, or reticulated structure. Wherein there is a total nun fiber of patches and any desired number of patches is separable from the total number of patches. Wherein there is a total number of patches and a desired number of patches in a desired shape, as viewed from above, is separable from the applicator. Wherein separation of patches is facilitated by markings or indicia on the applicator, by perforations on or through the applicator, by perforations around each patch, by perforations on or through each connector, by scoring around each patch, and/or by scoring on each connector. Wherein the applicator is marked with markings indicating a part of the applicator to be separated from the applicator for application to a particular body part or a particular skin area.

Wherein the treatment material is cosmetic material, attractant. material, repelling material, or de-scenting material. Including adhesive material on some or all of the patches, and/or adhesive material on some or all of the connectors. Including adhesive material only on the patches, with no adhesive material on the connectors. Including adhesive material only on the connectors, with no adhesive material on the patches, Including a release layer, the patches disposed removably on the release layer for selective removal of one patch or of a plurality of patches from the release layer.

Wherein the plurality of patches is a plurality of adjacent patches which remain adjacent to each other upon removal from the release layer. Wherein the connectors are string-like and the plurality of patches is two adjacent patches prior to removal from the release layer, such that upon removal from the release layer, the two previously adjacent patches are placeable on skin so that the two patches are at any desired angle to each other and are at any desired distance from each other. Wherein the connectors are string-like and the plurality of patches is two adjacent groups of multiple patches, the multiple patches in each group disposed with respect to each other in a set disposition and at a set distance from each other, such that upon removal from the release layer, the two groups of patches are placeable on skin so that the two groups of patches are at any desired angle to each other and are at any desired distance from each other, with the multiple patches in each group of patches remaining in the same disposition with respect to each other after removal from the release layer.

Wherein the connectors comprise a mesh or netting and the patches or on the mesh or netting, in one aspect the patches removable from the mesh or netting, and in another aspect the mesh or netting severable or cuttable so that one patch or a plurality of patches is selectively removable form the mesh or netting. Wherein the connectors comprise a chain mail structure and the patches are removably positioned on the chain mail structure. The connectors having a cross-sectional shape with ends connected to and between two adjacent patches and a middle between the ends, the ends with a height larger than a height of the middle.

The applicator of any of FIGS. 1A-2D, 5A, and 6-13B, or of any applicator herein with or without a release liner, with or without a release layer, with or without an opening in a layer for the application of or introduction of treatment material, with or without adhesive on each part, with or without adhesive on each patch, and/or with or without adhesive on each connector. Any applicator herein with or without a release liner, with or without a release layer, with or without an opening in a layer for the application of or introduction of treatment material, with or without adhesive on each part, with or without adhesive on each patch, and/or with or without adhesive on each connector. Any applicator herein wherein the connectors are formed integrally of the patches, formed separately from the patches and attached or connected thereto, and/or connected to patches by glue, cement, bonding, and/or sintering.

The present invention provides, in certain aspects and embodiments, a method for applying treatment material, the method including placing an applicator on skin, the applicator comprising any applicator according to the present invention, the applicator including treatment material. Such a method may include one or some, in any possible combination, of the following: for any applicator with an opening of a patch for the application of or for the introduction of treatment material, putting treatment material at the opening for introduction into the applicator; sealing the opening of a patch after the treatment material is put at the opening or introduced through the opening; separating a number of patches from the applicator, the number being a number less than the number of the total number of patches of the patches of the applicator; wherein separation is done by severing, tearing, breaking or cutting connectors; wherein the separation is done by severing, tearing, breaking, or cutting patches; wherein the separation is done by severing, tearing, breaking, or cutting both patches and connectors. Including one of: adhering patches to the skin, adhering connectors to the skin, and adhering patches to the skin and adhering connectors to the skin.

Wherein the treatment material is medicine, essential oil, a drug. Wherein the treatment material is one of perfume, repellant material, attractant material, and de-scenting material.

The present invention provides, in certain embodiments and aspects, a bandage including a plurality of parts, a plurality of connectors, a plurality of open spaces between the parts and the connectors, each part of the plurality of parts connected to adjacent parts by a connector or connectors of the plurality of connectors, each part of the plurality of parts spaced apart from adjacent parts by connectors between the parts, the open spaces having boundaries defined by edges of the parts and edges of the connectors, each part made of bandage material including, but not limited to, one of or a combination of cloth, cotton, gauze, absorbent material, textile material, woven material, dressing material, absorbent material, fabric, and nonwoven material.

Such a bandage may have one, or some, in any possible combination, of the following: wherein at least one or a plurality of the patches is suitable for use to apply treatment material to skin; wherein the patches are as any patch disclosed herein; wherein the patches, the connectors, or both have or are made of nonadherent material that does not adhere to skin; wherein the patches, the connectors, or both have or are made of absorbent dressing material; and/or wherein the connectors are as any connector disclosed herein. The present invention discloses methods for bandaging, the method comprising bandaging with a bandage, the bandage being any disclosed herein, and any applicator herein usable as a bandage.

The present invention discloses dressings for dressing for a wound, the dressing in one aspect being any applicator herein usable as a dressing and/or including a plurality of parts, a plurality of connectors, a plurality of open spaces between the parts and the connectors, each part of the plurality of parts connected to adjacent parts by a connector or connectors of the plurality of connectors, each part of the plurality of parts spaced apart from adjacent parts by connectors between the parts, the open spaces having boundaries defined by edges of the parts and edges of the connectors, and each part made of wound dressing material. Such a dressing may include one or some of the following, in any possible combination: wherein at least one or a plurality of the patches is suitable for use to apply treatment material to skin; wherein the patches are as any patch disclosed herein; and/or the connectors are as any connector disclosed herein. The present invention disclosed, in certain aspects, methods for dressing a wound, the methods including dressing the wound with a dressing, the dressing being any disclosed herein.

The present invention discloses a subunit patch including: a removable protective layer, a matrix layer containing treatment material which, in one particular aspect, is a physiologically effective amount of an analgesic, for example, but not limited to mepivacaine or buprenorphine, an adhesive layer, and a cover layer. The present invention discloses a method for treating or alleviating pain, including: applying a main patch or part thereof, according to any disclosed herein, to a patient in need thereof.

The present invention discloses a process for preparing a subunit patch, including the following steps: providing a composition comprising treatment material, and a matrix layer with the composition, providing an adhesive composition and producing an adhesive layer therefrom, providing a protective layer, providing a cover layer, and producing a subunit patch as any disclosed herein.

The present invention discloses an applicator wherein the applicator has an applicator top surface area, the patches combined have a patches top surface area, the connectors combined have a connectors top surface area, and the patches top surface area is about 70% of the applicator top surface area. The present invention discloses an applicator wherein the applicator has an applicator top surface area, the patches combined have a patches top surface area, the connectors combined have a connectors top surface area, and the connectors top surface area is about 10% of the applicator top surface area. The present invention discloses an applicator wherein the applicator has an applicator top surface area, the patches combined have a. patches top surface area, the connectors combined have a connectors top surface area, between patches and connectors there are open spaces and the area of the combined open spaces is about 20% of the applicator top surface area.

As can be seen from the foregoing, the aspects, concepts, features, and elements of the present invention may be embodied in a variety of structures, a variety of patches, and its methods done in a variety of ways. It involves structures, systems, method steps, and techniques to accomplish the appropriate ends and desired goals. Techniques and method steps according to the present invention are disclosed as part of the results to be achieved by the various strictures and described and as steps which for utilization of the structures as intended and described. In addition, while some structures are disclosed, it should be understood that these not only accomplish certain methods, but also can be varied in a number of ways within the scope of the present invention. It should also be understood that a variety of changes may be made without departing from the scope of the invention. Such changes are also implicitly included in the description and still fall within the spirit and scope of this invention.

I claim:

1. An applicator for applying treatment material, the applicator comprising:
a plurality of patches; and
a plurality of connectors;
each patch of the plurality of patches connected to adjacent patches by a connector or connectors of the plurality of connectors;
each patch for the application of treatment material to skin.

2. The applicator of claim 1 further comprising treatment material in or on one or more of the plurality of patches.

3. The applicator of claim 1 wherein each patch of the plurality of patches is generally sized and shaped the same and the plurality of patches is arranged in a regular array.

4. The applicator of claim 1 wherein at least one patch of the plurality of patches comprises a layered material.

5. The applicator of claim 1 wherein at least one patch of the plurality of patches defines at least one aperture therethrough, the at least one aperture positioned to allow the introduction of treatment material therethrough.

6. The applicator of claim 1 further comprising an adhesive material in or on one or more of the plurality of patches and/or in or on one or more of the plurality of connectors.

7. The applicator of claim 1 further comprising a release layer, the patches disposed removably on the release layer for selective removal of one or more patches from the release layer.

8. The applicator of claim 1 wherein the plurality of connectors comprises flexible connectors, wherein the flexible connectors allow connected patches to be positioned adjacent each other or separated from each other.

9. The applicator of claim 1 wherein a subgroup of the plurality of patches may be separated from a remainder of the plurality of patches.

10. The applicator of claim 2 wherein the treatment material is one of or a combination of any two of or of any three of: medicine, drugs, essential oils, attractants, repellants, descenters, perfumes, sterilizers, cleansers, anesthetics, natural supplements, herbal products, and/or any pharmacologically active substance that imparts local and/or systemic effects.

11. The applicator of claim 1 Wherein one or more patches of the plurality of patches is made of one of or a combination of woven material or of nonwoven material;
the woven material including but not limited to cloths, fabrics, textiles, linen, gauze, cotton, dressing material, barrier material, synthetic fabrics, adhesive substances, latex, natural or synthetic rubber, elastic, compounds or structures with elastic properties, plastics, bio-synthetics, cell-based sheets, or any combination of two or three of these or more;
the nonwoven material including but not limited to air laid material, felts, pads, mats, and material with components bonded, glued, cemented, fused, and/or sintered together.

12. The applicator of claim 1 wherein one or more connector of the plurality of connectors is made of one of or a combination of woven material or of nonwoven material;
the woven material including but not limited to cloths, fabrics, textiles, linen, gauze, cotton, dressing material, barrier material, synthetic fabrics, adhesive substances, latex, natural or synthetic rubber, elastic, compounds or structures with elastic properties, plastics, bio-synthetics, cell-based sheets, or any combination of two or three of these or more;
the nonwoven material including but not limited to air laid material, felts, pads, mats, and material with components bonded, glued, cemented, fused, and/or sintered together.

13. The applicator of claim 1 wherein one or more connector of the plurality of connectors is made of string-like material, including, but not limited to fibers, fibrils, and fibrous material.

14. An applicator for dermal or transdermal application of treatment material, the applicator comprising:
a plurality of sub-unit patches, each sub-unit patch of the plurality of patches having a common size and geometric shape, the plurality of sub-unit patches arranged in a regular array;
a plurality of connectors positioned between and connecting each of the sub-unit patches to adjacent sub-unit patches in the regular array, the plurality of connectors comprising flexible material, the flexible connectors allowing adjacent sub-unit patches to be positioned adjacent each other or separated from each other;
adhesive material positioned on at least one sub-unit patch and/or at least one connector; and
a release liner positioned over the plurality of sub-unit patches and the plurality of connectors, covering at least the adhesive material;
each sub-unit patch for the application of treatment material to skin.

15. The applicator of claim 14 wherein a subgroup of the plurality of sub-unit patches may be separated from a remainder of the plurality of sub-unit patches.

16. The applicator of claim 15 wherein the separation of the subgroup of sub-unit patches is facilitated by markings or indicia on the applicator, by perforations on or through the applicator, by perforations around each sub-unit patch, by perforations on or through each connector, by scoring around each sub-unit patch, and/or by scoring on each connector.

17. A method for applying treatment material, the method comprising:
providing a multi-patch array comprising a plurality of patches and a plurality of connectors, each patch of the plurality of patches connected to adjacent patches by a connector or connectors of the plurality of connectors;
placing treatment material in or on at least one of the plurality of patches; and
positioning the multi-patch array on the area to be treated.

18. The method of claim 17 further comprising the step of separating a subgroup of patches from the remainder of the plurality of patches and the step of positioning the multi-patch array comprises positioning the subgroup of patches.

19. The method of claim 17 further comprising the step of placing adhesive material in or on one or more of the plurality of patches and/or in or on one or more of the plurality of connectors and the step of positioning the multi-patch array further comprises positioning at least a portion of the adhesive material proximate to the area to be treated.

20. The method of claim 17 wherein at least one patch in the multi-patch array defines at least one aperture therethrough and the method further comprises the step of introducing treatment material into and through the at least one aperture.

21. A bandage comprising:
a plurality of parts; and
a plurality of connectors;
each part of the plurality of parts connected to adjacent parts by a connector or connectors of the plurality of connectors, a plurality of open spaces formed between the parts and the connectors, the open spaces having boundaries defined by edges of the parts and edges of the connectors;
each part made of bandage material including, but not limited to, one of or a combination of cloth, cotton, gauze, absorbent material, textile material, woven material, dressing material, absorbent material, fabric, and nonwoven material.

22. The bandage of claim 21 wherein at least one of the plurality of parts comprises treatment material.

23. The bandage of claim 21 wherein at least one of the plurality of parts and/or the plurality of connectors comprises absorbent dressing material.
